(19) United States
(12) Patent Application Publication
(10) Pub. No.: US 2013/0231372 A1
(43) Pub. Date: Sep. 5, 2013

(54) Small Molecule Inhibitors Of P-type ATPases

(71) Applicants: Mary Matsui, Teaneck, NJ (US); Daniel Yarosh, Merrick, NY (US)

(72) Inventors: Mary Matsui, Teaneck, NJ (US); Daniel Yarosh, Merrick, NY (US)

(21) Appl. No.: 13/409,372

(22) Filed: Mar. 1, 2012

(51) Int. Cl. A61K 31/4439; A61Q 19/00; C07D 401/04; A61K 8/49

(52) U.S. Cl. CPC A61K 31/4439; A61K 8/4946; A61Q 19/00; C07D 401/04
USPC 514/338; 546/273.7

(57) ABSTRACT

Small, low molecular weight compounds of the formula I and/or the formula II, as defined herein, which inhibit Cu-ATPases, ATP7A and ATP7B. Compositions and methods therefore, are provided.

### FIELD OF THE INVENTION

The present invention is generally concerned with inhibitors of P-type ATPases. More specifically, the present invention relates to small molecule inhibitors of the copper-transporting ATPases, ATP7A and ATP7B.

### BACKGROUND OF THE INVENTION

ATPases play numerous roles in human cell biology. Different types of ATPases can differ in function, structure, and the type of materials they transport. Many members of the P-type ATPases function to transport a variety of different ions across membranes in the cell. The ion gradients generated and maintained by these transporters, using ATP hydrolysis for energy, are utilized for nutrient uptake, propagation of electrical signals, and muscle contraction. Unlike many members of the P-type ATPase family, the subfamily of the P-type ATPases involved in the transport of copper does not generate ion gradients, since copper stays bound to proteins both in the cytosol and outside of the cell (Lutsenko, et al., 2008). The functions of these copper transporting ATPases (Cu-ATPases) include absorption of dietary copper, transfer of copper to the central nervous system for normal development and function of the central nervous system, and regulation of copper homeostasis throughout the body.

Copper is a micronutrient required by all organisms to maintain life. It serves as a co-factor for enzymes that catalyze a diverse array of essential biochemical reactions. Many physiological processes rely on proper supply of cells with copper. Nevertheless, excess levels of copper have been shown to be cytotoxic. Thus, it is not surprising that a network of copper-binding proteins (i.e., a system of chaperones and transporters) has evolved to carry and transport copper to various cellular destinations to maintain copper homeostasis. The presence of Cu-ATPases in all eukaryotic and prokaryotic cells has been established. Human cells express two homologous Cu-ATPases, ATP7A and ATP7B, which exhibit around 60% amino acid sequence identity, and are both localized primarily in a trans-Golgi compartment (Lutsenko, et al., 2008). To maintain copper homeostasis, these ion transporters couple ATP hydrolysis to the transport of copper from the cytosol across cellular membranes, exporting the copper to the bloodstream or out of the body, thus decreasing cytosolic copper concentration. Additionally, the Cu-ATPases are essential for the transport of copper from the cytosol into the lumen of intracellular compartments in the secretory pathway, including the trans-Golgi network and melanosomes, where copper is incorporated into copper-requiring enzymes (Yamaguchi, et al., 1996; Harris, 2000; Thiele, 2003). ATP7A delivers copper to enzymes, including tyrosinase, which is essential for melanogenesis; lysyl oxidase, which is necessary for the structure and function of bone, skin, hair, blood vessels and nervous system; and dopamine β-hydroxylase, which converts dopamine to norepinephrine; while ceruloplasmin, which carries copper in the blood and plays a role in iron metabolism, accepts copper from ATP7B (Petris, et al., 2000; D'Amico, et al., 2005).

The importance of copper as an essential nutrient is evident from the existence of disease phenotypes in humans caused by the inactivation of the copper transporters. Abnormalities in copper metabolism affect multiple organs with major impact on the central nervous system. Menkes Disease results from mutations or deletions in the gene encoding ATP7A. Individuals affected with Menkes Disease suffer from severe copper deficiency due to reduced absorption of dietary copper and defective distribution of copper to copper-dependent proteins within the body, and manifest severe neurological symptoms and developmental delays as well as vascular abnormalities, lung vasculature defects and poor muscle tone. The lack of copper delivery to the enzyme tyrosinase also results in a noticeable loss of pigmentation. (Menkes, et al., 1962; Kodama, et al., 1999). It is believed that human Cu-ATPases receive copper from a small cytosolic protein chaperone called Atox1. Deletion of the Atox1 gene in mice is associated with various symptoms including hypopigmentation (Hamza, et al., 2001; Ralle, et al., 1980).

Up-regulation of ATP7A has been associated with Alzheimer's disease (Zheng, et al., 2010) and with resistance to cancer chemotherapy (Leonhardt, et al., 2009).

Symptoms of Wilson's Disease manifest due to the inactivation of ATP7B. The abnormal accumulation of copper in the liver and the low levels of ceruloplasmin cause the hepatic and neurological symptoms of this disease (Wilson, 1912).

Human Cu-ATPases are large transmembrane proteins, each consisting of about 1500 residues. ATP7A is glycosylated, while ATP7B is not. Both proteins are phosphorylated. A schematic 3D protein structure of ATP7A (the structure of ATP7B is similar) with its functionally important domains is shown in FIG. 1, a schematic view of the copper ion (Cu⁺²) transporter protein ATP7A (Zeynep T., et al., 2010). Both ATP7A and ATP7B contain 8 transmembrane segments (TMS) that form intramembrane copper-binding sites, and N- and C-termini of the protein which are both oriented toward the cytosol. The N-terminus contains six homologous metal-binding domains (MBDs). Each of the six homologous MBDs comprises two invariant cysteines which bind a single copper ion from the cytosol (DiDonato, et al., 1997; Lutsenko, et al., 1997; Lutsenko, et al., 2007B; Yatsunyk, et al., 2007). The P, N and A sites are responsible for ATP binding, hydrolysis, phosphorylation and dephosphorylation. The A domain of Cu-ATPases is conserved among P-type ATPases. The P domain structure also is conserved between Cu-ATPases and among all members of the P-type ATPase family reflecting the common enzymatic mechanism through which all P-type ATPases operate, while the N-domain is unique for the Cu-ATPases (Sazinsky, et al., 2006; Dimitriev, et al., 2006). Residues at the N-terminus of the protein are involved in the apical targeting of ATP7B in hepatocytes. The C-terminal domains of ATP7A and ATP7B include di- and tri-leucine motifs, respectively, which may determine the targeting of membranes proteins to distinct membrane compartments and regulate the rate of their trafficking from and between the endosomal compartments. (Petris, et al., 1999; Francis, et al., 1999; Lane, et al., 2004). Cu-ATPases transport copper by binding ATP and copper from the cytosolic side of the membrane. ATP is hydrolyzed, phosphorylating the enzyme. The ATPase undergoes cycles of conformational changes that drive the passage of the metal across the membrane. Copper is released at the opposite side of the membrane. The ATPase is dephosphorylated and the ATPase returns to its initial state for re-initiation of the cycle (Voskoboinik, et al., 1999; Voskoboinik, et al., 2001; Mandal, et al., 2003; Barnes, et al., 2005; Hung, et al., 2007). As it has been observed that the rate of copper transport by ATP7B appears to increase at lower pH, it has been suggested that the lower pH, common to intracellular compartments in which Cu-ATPases operate, may facilitate copper dissociation from the transporter and stimulate metal transport (Lutsenko, et al. 2008).

It has been established that under low copper (basal) conditions, both ATP7A and ATP7B reside in the trans-Golgi network (TGN). The current model is one of constitutive recycling of ATP7A between the TGN, vesicles in close vicinity to the plasma membrane, and the plasma membrane, under basal conditions. The introduction of copper to cells is a recognized means by which to study the trafficking of the protein. When copper is introduced, the steady state distribution of ATP7A and ATP7B is shifted toward the vesicles and the cell surface (Mercer, et al. 2003 and Guo, et al. 2005). ATP7A relocalizes towards the basolateral membrane to facilitate copper export into blood (Greenough et al., 2004), while ATP7B, in hepatic cells, traffics towards the apical canalicular membrane to transport copper into the bile (Schaefer, et al., 1999). More specifically, when copper levels are elevated, ATP7A traffics to the vesicles near the plasma membrane where excess copper is delivered into the lumen of the vesicles. The vesicles fuse with the plasma membrane which releases copper into the extracellular environment. ATP7A is endocytosed and returns to a recycling vesicular compartment. In hepatocytes, where ATP7B is expressed and ATP7A is absent, it has been observed that the mechanism of trafficking of ATP7B is similar to that of ATP7A (Schaefer, et al., 1999; Roelofsen, et al., 2000; Guo, et al., 2005; and Bartee et al., 2007). The relocalization of ATP7A and ATP7B is reversible once copper has been removed from culture media.

Recent studies have implicated ATP7A and ATP7B in the resistance to the anti-cancer drug, cisplatin (cis-diamminedichloroplatinum or DDP), which has been used in chemotherapy for various cancerous tumors, and particularly in the treatment of testicular and ovarian cancers. Cisplatin reacts with nuclear DNA and prevents normal replication which affects rapidly dividing cancer cells. Eventually, as is the case with most anticancer drugs, patients develop drug resistant cells which do not respond to this therapy. In the cell, copper is transported by the chaperone protein, Atox1. It has been shown that cisplatin binds to Atox 1 and that the platinum-binding site is the same as the copper-bind site (Wernimont, 2000; Boal, 2009). It was also reported that a higher level of ATP7B expression is often associated with tumor resistance to cisplatin (Komatsu, 2000; Nakagawa 2008). Silencing ATP7B expression was associated with decreased cell survival in the presence of cisplatin (Yoshizawa, 2007) while an increase in ATP7A expression correlated with increased tumor resistance to cisplatin (Samimi, 2004).

Kalayda, et al. (2008) studied the effect of the copper transporters, ATP7A and ATP7B, in the anti-cancer drug resistance of cell lines molecularly engineered to express either ATP7A or ATP7B. In drug-sensitive cells, both transporters appeared to undergo constitutive recycling between the trans-Golgi network and more peripherally located vesicles in the cytosol (similar to the system of copper-induced trafficking of these transporter proteins to maintain copper homeostasis). Cisplatin exposure was observed to trigger rapid trafficking of both transporters from the Golgi toward the cell periphery, thus suggesting that the cells utilize the same vesicular export pathway for efflux of cisplatin as they employ for export of copper. In a cisplatin-resistant cell line; however, continuous recycling of the copper efflux transporters appeared to be blocked. Studies indicated that, given the higher expression of this transporter, ATP7A most likely mediates sequestration (rather than efflux) of the drug in the cisplatin-resistant cells, while re-localization of ATP7B away from the trans-Golgi to peripherally located vesicles appears to prevent cisplatin binding to this protein and therefore efflux of the drug.

Understanding the trafficking of ATP7A and ATP7B under basal and copper-stimulated conditions is therefore essential to elucidating the role of these proteins in delivering copper to copper-dependent enzymes and the maintenance of copper homeostasis via copper efflux. Using this model, and modifying or inhibiting the activity of the copper-transporters, may also have implications in various treatments and therapies for ATP7A- and/or ATP7B-related conditions, e.g., for treatment of hyperpigmentation of skin, Alzheimer's disease, anti-cancer drug-resistance, and so forth. There is therefore a need for small molecular weight inhibitors of ATP7A and ATP7B, which are capable of penetrating cells, for use in therapeutic regimens.

### SUMMARY OF THE INVENTION

The present invention relates to safe and effective compounds and compositions which modulate the activity of the Cu⁺² transporters, ATP7A and/or ATP7B. More specifically, the invention pertains to certain small, low molecular weight compounds, known as 2-pyridylmethylsulfinyl-benzimidazoles and substituted imidazopyridines, also known as proton pump inhibitors or PPIs, which inhibit the cellular activity of P-type Cu-ATPases, ATP7A and/or ATP7B. The invention also pertains to methods of inhibiting ATP7A and/or ATP7B to treat various conditions, including hyperpigmentation of the skin, Alzheimer's disease, anti-cancer drug-resistance, and so forth. Also disclosed are certain derivatives of the PPI, omeprazole.

### BRIEF DESCRIPTION OF THE FIGURES

FIG. 1 is a schematic view of the copper ion (Cu⁺²) transporter protein ATP7A.

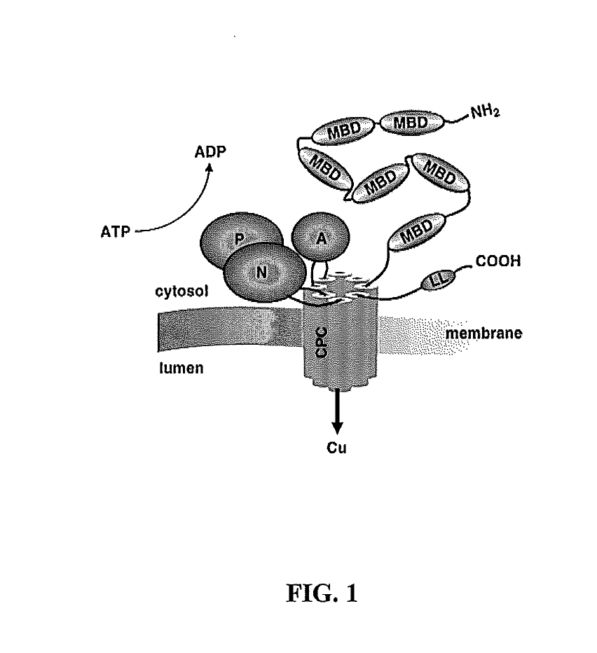

FIG. 2 represents light microscopic photographs of in situ tyrosinase activity in omeprazole-(OPZ-) treated B16F10 mouse melanoma cells.

FIG. 3 represents a comparison of partial amino acid sequences of proteins ATP4A and ATP7A, showing the known and putative binding sites for omeprazole and SCH-28080 (SCH).

FIG. 4 represents images of co-localization assays, using fluorescent markers for Golgi complex and ATP7A, showing Cu+²-stimulated ATP7A trafficking.

FIG. 5 represents light microscopic photographs of in situ tyrosinase activity in Me32a Menkes disease fibroblasts.

FIG. 6 represents images of co-localization assays, using fluorescent markers for Golgi complex and ATP7A, showing SCH-28080 inhibition of Cu+²-stimulated ATP7A re-localization in B16F10 mouse melanoma cells.

FIG. 7 represents a Western gel analysis followed by in situ tyrosinase activity in B16F10 mouse melanoma cells, demonstrating Cu⁺² rescue of cells from low levels of omeprazole.

FIG. 8 represents Human HepG2 (hepatoma) cells stably transfected with a myc-tagged form of ATP7B, demonstrating inhibition of trafficking of Cu+² transporter protein ATP7B by omeprazole.

### DETAILED DESCRIPTION OF THE PREFERRED EMBODIMENTS OF THE INVENTION

The inventors took advantage of their previous observations that a class of compounds identified as Type I H+, K+ ATPase inhibitors (PPIs), the substituted-benzimidazoles, e.g., omeprazole and its various analogues, and also, structurally related compounds including substituted imidazopyridines, such as SCH-20808, all strongly inhibited melanogenesis (see U.S. Published Appln. No.: 2011/0171149, Jul. 14, 2011, which is herein incorporated by reference in its entirety). PPIs had been known to function in the parietal cells to block the production of stomach acid by binding to at least one cysteine residue of the gastric proton pump, ATP4A, to thus effect long-lasting, semi-irreversible inactivation of the ATP4A. In order to bind to the ATPase, the PPI must be in its active form, and the PPI requires an acidic environment to undergo rearrangement to its active form. The inventors further observed that another gastric proton pump inhibitor, a substituted-imidazopyridine compound, SCH-28080, having a different reactive site on ATP4A in parietal cells, also inhibited melanogenesis. Because both omeprazole (and its analogues) and structurally related compounds, such as SCH-28080, inhibit melanogenesis despite differences in structure and mechanisms of action, the inventors were led to the novel generalization that inhibitors of Type I H⁺, K⁺-ATPases are also inhibitors of melanogenesis, and that these inhibitor compounds could be useful in depigmenting skin. In fact, the inventors determined that omeprazole reduced cellular melanin content with low cytotoxicity and inhibited melanogenesis with an EC₅₀ of 15 μg/ml in normal human epidermal melanocytes, and in a reconstructed human skin model, omeprazole was effective at 50 μg/ml. In a preliminary clinical test, 0.0035% omeprazole was shown to significantly reduce skin pigment levels after 3 weeks.

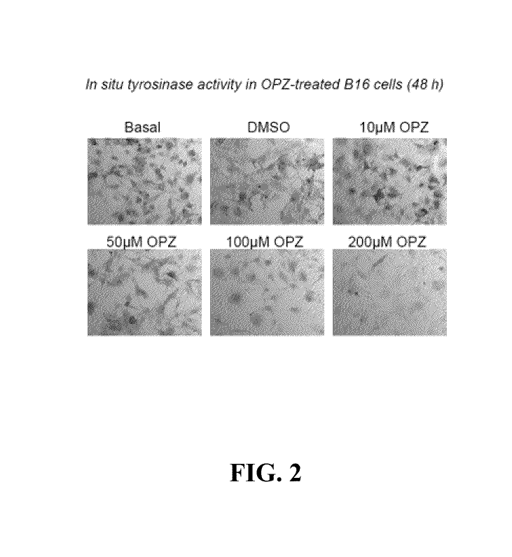

The inventors next examined the effect of omeprazole on tyrosinase, the principal, rate-limiting enzyme in melanin synthesis. It was found that omeprazole had no significant direct effect on human tyrosinase activity, since it had been determined that, while 250 μg/ml of omeprazole is required to moderately inhibit human tyrosinase activity using extracts from human melanocytes, just 5-50 μg/ml of omeprazole inhibits melanogenesis in cultured melanocytes. Additionally, it was observed, using an assay for tyrosinase with L-DOPA as a substrate and measuring the formation of the brown pigment, DOPA-chrome, that although omeprazole only slightly decreased tyrosinase DOPA oxidase activity of mushroom and B16F10 cell extract in vitro, the in situ tyrosine hydroxylase activity of tyrosinase was decreased significantly in omeprazole-treated B16F10 cells (see FIG. 2). Furthermore, it was demonstrated that omeprazole had no effect on tyrosinase mRNA levels, as it did not alter mRNA levels of other melanogenesis related proteins such as MITF, TRP-2, and Pmel17 when B16F10 mouse melanoma cells were treated with omeprazole and the levels of tyrosinase or MITF or TRP-2, and Pmel17-specific mRNA was measured in total extracted RNA using gene-specific complementary primers and RT-PCR. Further observations by the inventors were that omeprazole significantly decreased total tyrosinase protein level, as determined by analysis with Western blots using antibodies specific for mouse tyrosinase, suggesting reduced translation or increased degradation, possibly due to destabilization of the protein, and that omeprazole treatment of B16F10 cells resulted in a decrease in the ratio of mature to immature glycosylated tyrosinase as determined using the method of carbohydrate cleavage by EndoH and PNGaseF treatment followed by Western-blotting (Wang et al., 2005).

Taken together, these results suggested to the inventors that omeprazole decreases the level of functional tyrosinase protein in melanocytes. The inventors then investigated whether the Type I H⁺, K⁺-ATPase, ATP4A, which is the target of omeprazole in the gastric lumen, was also found in melanocytes. It was determined by analysis with rtPCR using specific TaqMan MGB probes for ATP4A, ATP7A and ATP12A, that ATP4A is expressed below the level of detection in both B16F10 mouse melanoma cells or normal human melanocyte and therefore cannot be the target of omeprazole in these cells.

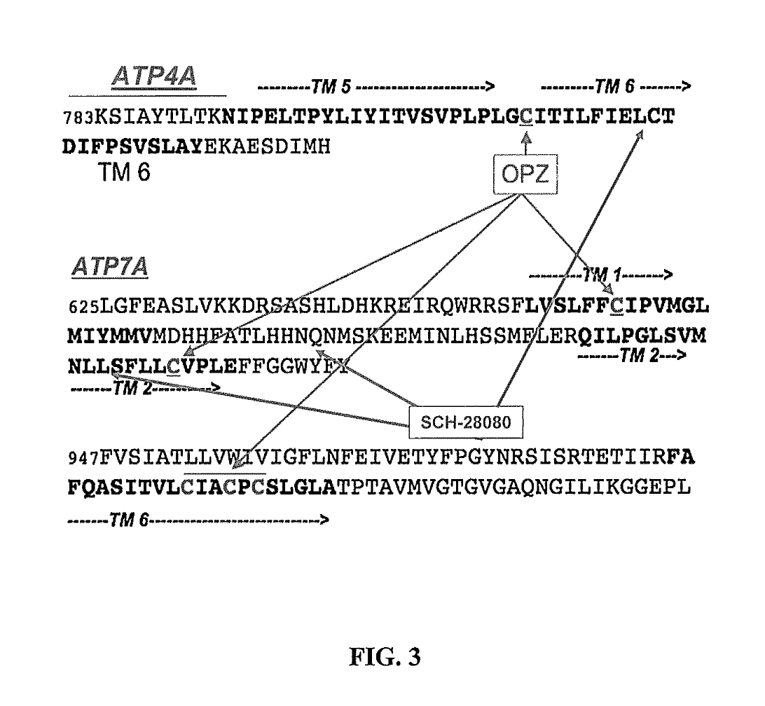

The inventors then considered other P-type ATPases, ATP7A and ATP12A, having at least partial sequence homology with ATP4A (i.e., including one or more cysteine residues) as the possible targets of omeprazole. The inventors considered both ATP7A and ATP12A which are found in normal human epidermal melanocytes (NHEM) and B16F10 mouse melanoma cells. However, as it was known that ouabain inhibits ATP12A, but the inventors determined that ouabain does not inhibit melanogenesis, ATP12A was eliminated as a likely target. As FIG. 3 shows, ATP4A (upper amino acid sequence) has a known binding site for omeprazole (the cysteine represented by “C”). ATP7A (lower amino acid sequence), similarly to ATP4A, also has cysteine residues in transmembrane regions (at least TM 1, 2 and 6) of the protein available for omeprazole binding, suggesting that, under certain conditions, omeprazole may be able to bind to ATP7A. The inventors hypothesize, by analogy to its binding locus in ATP4A, that SCH28080 also binds inside channels formed by the ATP7A transmembrane regions. Consequently, the inventors proceeded to further examine ATP7A for a possible role in the melanocyte response to omeprazole.

The consideration of ATP7A and ATP7B as targets for PPIs is novel. Despite the great interest in ATP7A and ATP7B, no small molecule inhibitors of any type are available. No consideration of PPIs as inhibitors of ATP7A or ATP7B had been previously reported.

The inventors surprisingly and unexpectedly have discovered that small compounds which inhibit P-type ATPases, having the structural formula I or the structural formula II, shown below, also inhibit ATP7A and/or ATP7B. The inventors found that they could alter the trafficking of ATP7A and/or ATP7B, and thus interfere with Cu⁺² delivery to Cu⁺²-dependent enzymes. Therefore, the first aspect of the present invention concerns a small molecule inhibitor of ATP7A and/or ATP7B having the formula:

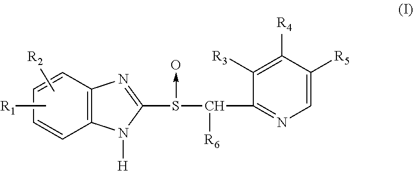

wherein:
R₁ and R₂ are same or different and are each selected from the group consisting of hydrogen, alkyl, carbomethoxy, carboethoxy, alkoxy, and alkanoyl, any of which may be halogen-substituted, and halogen;
R₆ is selected from the group consisting of hydrogen, methyl, and ethyl; and
R₃, R₄ and R₅ are the same or different and are each selected from the group consisting of hydrogen, methyl, methoxy, ethoxy, methoxyethoxy, ethoxyethoxy, propoxy, propoxymethoxy, and the like, any of which may be halogen-substituted; or a derivative or physiologically acceptable salt, solvate or bioprecursor, or stereoisomer or enantiomer thereof;
   or having the formula:

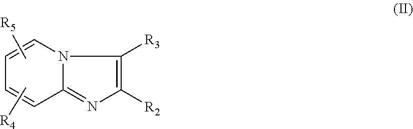

wherein:
R₂ is hydrogen, lower alkyl or hydroxy lower alkyl;
R₃ is lower alkyl, —CH₂CN, hydroxy lower alkyl, —NO, —CH₂N═C or

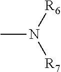

(wherein R₆ and R₇ are independently selected from the group consisting of hydrogen and lower alkyl) or hydrogen provided R₂ is not hydrogen;
R₄ is Z-T-W wherein Z represents —O—, —NH— or a single bond; T represents a straight- or branched-chain lower-alkylene group; when Z is a single bond, T also represents an ethenylene or a propenylene group wherein the unsaturated carbon is at the single bond; when Z is —O—, T also represents an allylene group wherein the saturated carbon is at the oxygen; and W represents hydrogen, when T is allylene and Z is —O—, and Ar, wherein Ar is selected from thienyl, pyridinyl, furanyl, phenyl and substituted phenyl wherein there are one or more substituents on the phenyl independently selected from halogen or lower alkyl; and
R₅ is hydrogen, halogen or lower alkyl; or a derivative or physiologically acceptable salt, solvate or bioprecursor, or stereoisomer or enantiomer thereof.

Non-limiting examples of compounds of formula I include: omeprazole, 5- or 6-methoxy-2-{[(4-methoxy-3,5-dimethylpyridin-2-yl)methyl]sulfinyl}-1H-benzimidazole; esomeprazole, S-5-methoxy-2-{(4-methoxy-3,5 dimethylpyridin-2-yl)methylsufinyl]-3H-benzoimidazole; lansoprazole, 2-{[3-methyl-4-(2,2,2-trifluoroethoxy)pyridin-2-yl]methylsulfinyl-1H-benzo(d)imidazole; pantoprazole, RS-6-(difluoromethoxy))-2-[(3,4-dimethoxypyridin-2-yl)methylsulfinyl]-1H-benzo(d)imidazole; rabeprazole (pariprazole), 2-([4-(3-methoxypropoxy)-3-methylpyridin-2-yl]methylsulfinyl)-1H-benzo(d)imidazole, leminoprazole, 2-((o-(isobutylmethylamino)benzyl)sulfinyl)benzimidazole; and timoprazole, 2-(pyridine-2-ylmethylsulfinyl)-1H-benzimidazole. In a preferred embodiment of the present invention, the compound comprises omeprazole, 5- or 6-methoxy-2-{[(4-methoxy-3,5-dimethylpyridin-2-yl)methyl]sulfinyl}-1H-benzimidazole.

As compounds of formula I, specific mention may also be made of certain derivatives of omeprazole. Below neutral pHs (e.g., at acidic pHs), omeprazole reorganizes through intermediates into a sulfonamide analog. Omeprazole has 2 pKa values of 4.0 and 0.8, and has a sulfinyl group that is responsible for its activity but also for its reactivity and reorganization. At a pH below 0.8, omeprazole quickly reorganizes into the sulfonamide analog. Omeprazole derivatives that undergo partial rearrangement but still retain depigmentation activity may be prepared by acidifying omeprazole in the presence of a sulfur-containing compound, such as L-cysteine, L-cysteamine, 2-mercaptoethanol or glutathione, and the like. As one example, treatment of a mixture of omeprazole and L-cysteine in 1N HCl, preferably in a ratio of from about 1:1 to 1:2 omeprazole: L-cysteine, results in compounds 1, 2, 5 and 6 (see the scheme below). Compounds 5 and 6 have been shown by the inventors to inhibit the development of pigment in melanocytes (data not shown).

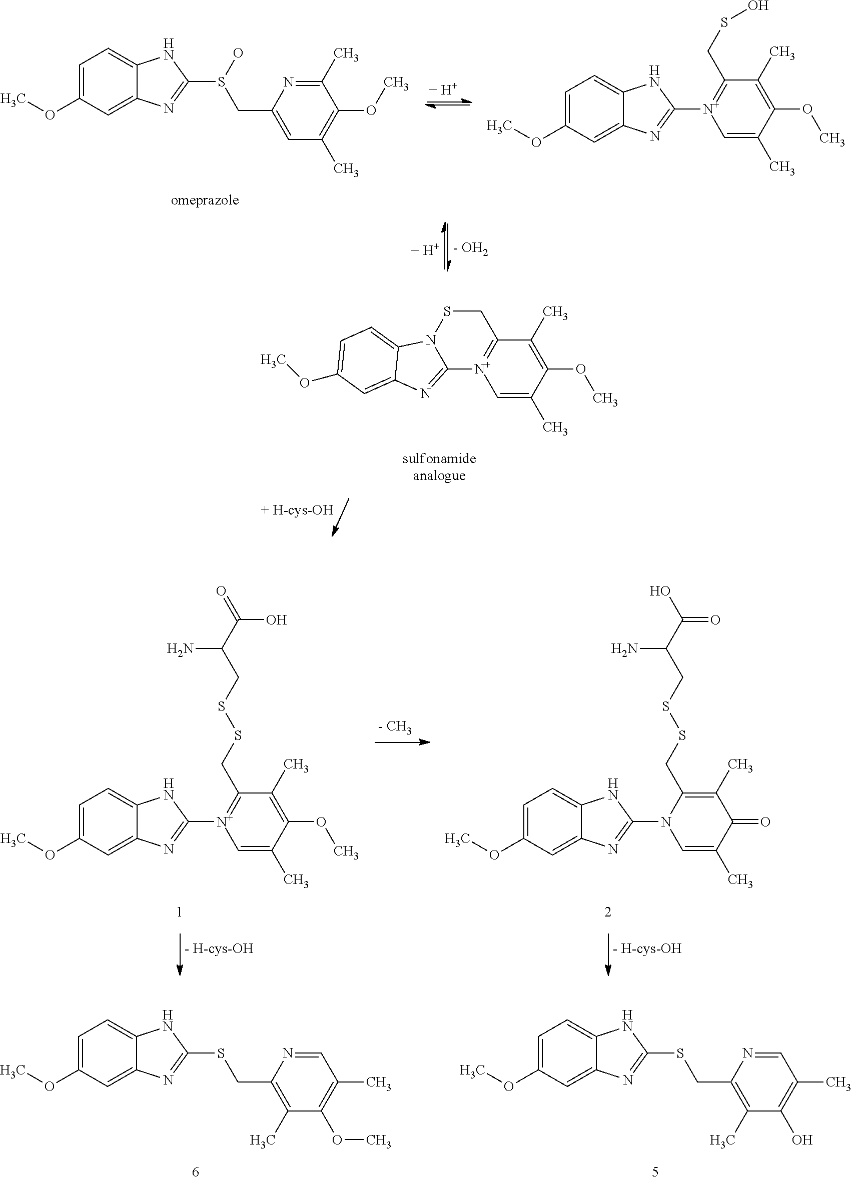

Non-limiting examples of compounds of formula II include: SCH-28080; soraprazan [(7R,8R,9R)-2,3-dimethyl-8-hydroxy-7(2-methoxyethoxy)-9-phenyl-7,8,9,10-tetrahydro-imidazo-[1,2-h][1,7]-naphthyridine], pumaprazole 8-(2-methoxycarbonylamino-6-benzulamino)-2,3-dimethylimidazo-[1,2-a)pyridine-D,L-hemimalate, AR-H047108, (8-[(2-ethyl-6-methylbenzyl)amino]2,3-dimethylimidazo[1,2-a]pyridine-6-carboxamide; dapiprazole, 3-{2-[4-(2-methylphenyl)piperazin-1-yl]ethyl}-5,6,7,8-tetrahydro-[1,2,4]triazolo[4,5-a]pyridine; AZD0865, ((8-[2,6-dimethylbenzyl)amino]-N-(2-hydroxyethyl)-2,3-dimethylimidazo[1,2-a]pyridine-6-carboxamide; and tenatoprazole, 3-methoxy-8-[(4-methoxy-3,5-dimethyl-pyridin-2-yl)methylsulfinyl]-2,7,9-triazabicyclo[4.3.0]nona-2,4,8,10-tetraene. In a preferred embodiment of the present invention, the compound comprises SCH-28080.

In accordance with a second aspect of the present invention, there are provided cosmetic, dermatological and/or pharmaceutical compositions containing, comprising or consisting essentially of, a small molecule inhibitor of ATP7A and/or ATP7B, having the formula I or the formula II, in a cosmetically-, dermatologically- or pharmaceutically-acceptable vehicle, carrier or diluent therefor. By use of the term “consisting essentially of”, it is intended that the compositions of the invention exclude additional, unspecified components that would adversely affect the basic and novel characteristics imparted to the composition by the recited components.

The small molecule inhibitors or PPIs must be delivered to the melanocytes without passing through an acid compartment so oral administration is the least preferred method for this class of small molecule inhibitors of ATP7A or ATP7B.

Since only small amounts are needed to inhibit the ATP7A/B enzymes, the small molecule inhibitors should be administered close to the melanocytes to minimize exposure to other body organs and maximize safety; therefore subcutaneous injection is preferable to systemic injection. Topical application is the most preferred method.

PPIs from the omeprazole family are highly reactive in water, so they are preferably formulated as solids or in anhydrous or organic solvents, such as oils, alcohols or dimethylsulfoxide (DMSO) or in pharmaceutically acceptable oil-based carriers for injection. Other small molecules that are stable in water may be formulated in hydrous forms, such as emulsions, creams, lotions, gels, serums, toners or in pharmaceutically acceptable water-based carriers for injection.

Suitable compositions could contain ingredients that would be compatible with the formulation. Compositions should not include any, or more than low levels of, copper, since the object of the invention is to deny copper to copper-dependent enzymes, such as tyrosinase.

Suitable compositions could contain ingredients that enhanced the depigmentation effect, by acting on the tyrosinase or another step in the melanogenesis pathway. Such ingredients include Vitamin C and its derivatives, UP302, hydroquinone and its derivatives such as arbutin and mulberry extract, licorice extract, ellagic acid and ellagitannins, and others. They might also inhibit depigmentation by blocking or attenuating the agents that trigger melanogenesis, such as sunscreens to reduce UV and sun exposure, and anti-inflammatories such as ibuprofen, acetaminophen, green tea, chamomile, and the like, that reduce inflammation and lower levels of inflammatory signaling molecules. These ingredients which enhance the depigmentation effect can be included in the same formulation as the PPI or in a separate composition applied at the same time or in a temporal regimen together with the invention.

Suitable compositions could contain ingredients that reduce the aging process or the appearance of aged skin, such as collagen inducers like Vitamin C, and metalloproteinase I inhibitors such as extracts of Siegesbeckia. The compositions could also contain ingredients that improve the feel and texture of the skin, such as moisturizers, emollients, hydrators, exfoliators, smoothing agents, pore reducers, and the like.

Suitable compositions should be applied as often as necessary, the preferred regimen being once or twice a day. Since it takes some time for the inhibition of ATP7A or ATP7B to take effect, the benefits of the invention may not be apparent for 12 to 24 hours. In addition, because some of the PPIs may bind covalently to ATP7A or ATP7B, the effects may last for several hours or even days after withdrawal of the PPI.

The inhibitors of P-type ATPases may be used in a pharmaceutical product or a cosmetic or dermatological product. Skin compositions of the invention may comprise from about 0.00005% to about 0.5% of the active compound by weight of the total composition, more preferably from about 0.0005% to about 0.05%, more preferably still from about 0.001% to about 0.1%, such as about 0.0035%.

Cosmetic or dermatological compositions of the present inventions may be found in a variety of forms, such as anhydrous compositions, aqueous-based solutions, serums, gels, creams, lotions, mousses, sticks, sprays, ointments, essences, pastes, microcapsules, or color cosmetic compositions such as foundation, blush, eyeshadow, and the like. They may contain other additional cosmetically and/or dermatologically acceptable ingredients, such as additional skin lightening agents or tyrosinase inhibitors, antioxidants, anti-inflammatory agents, botanicals, humectants, moisturizers, sunscreens, preservatives, colorants, perfumes, and the like. In the case where the composition is in the anhydrous form the P-type ATPase inhibitor compound or derivative thereof may be solubilized or dispersed in the oil phase of the emulsion; or if the P-type ATPase inhibitor compound or derivative thereof is water soluble it may be solvated in polar solvents, typically ingredients referred to as humectants such as glycerine or alkylene glycols prior to formation of an anhydrous emulsion. If the composition is in the emulsion form, the P-type ATPase inhibitor compound or derivative thereof may be found in the water phase or the oil phase of the emulsion depending on the type of derivative. For example, certain hydrophilic derivatives which are water soluble will generally be solubilized in the water phase of the emulsion. Certain other derivatives which are lipophilic in nature will more likely be found in the oil phase of the emulsion.

Suitable serums or gels will generally comprise from about 1-99% water, and optionally from about 0.001-30% of an aqueous phase thickening agent. The other ingredients mentioned herein may be present in the percentage ranges set forth.

Typical skin creams or lotions comprise from about 5-98% water, 1-85% oil, and from about 0.1 to 20% of one or more surfactants. Preferably the surfactants are nonionic and may be in the form of silicones or organic nonionic surfactants.

Typical color cosmetic compositions such as foundations, blush, eyeshadow, and the like, will preferably contain from about 5-98% water, 1-85% oil, and from about 0.1 to 20% of one or more surfactants in addition to from about 0.1 to 65% of particulates which are pigments or a combination of pigments and powders.

In the case where the compositions are in the form of aqueous solutions, dispersions or emulsions, in addition to water, the aqueous phase may contain one or more aqueous phase structuring agents, that is, an agent that increases the viscosity, or thickens, the aqueous phase of the composition. This is particularly desirable when the composition is in the form of a serum or gel. The aqueous phase structuring agent should be compatible with the P-type ATPase inhibitor compound or derivative thereof, particularly if the particular P-type ATPase inhibitor compound or derivative thereof is water soluble, and also compatible with the other ingredients in the formulation. Suitable ranges of aqueous phase structuring agent, if present, are from about 0.01 to 30%, preferably from about 0.1 to 20%, more preferably from about 0.5 to 15% by weight of the total composition. Examples of such agents include various acrylate-based thickening agents, natural or synthetic gums, polysaccharides, and the like, including but not limited to those set forth below. When the P-type ATPase inhibitor compound or derivative thereof is in the water soluble form, the aqueous phase thickening agent also contributes to stabilizing this ingredient in the composition and improving penetration into the stratum corneum. Such structuring agents may include the following:

### A. Polysaccharides

Polysaccharides may be suitable aqueous phase thickening agents. Examples of such polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

### B. Acrylate Polymers

Also suitable are different types of synthetic polymeric thickeners. One type includes acrylic polymeric thickeners comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof are suitable. In one embodiment the A monomer comprises one or more of acrylic acid or methacrylic acid, and the B monomer is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. The acrylic copolymer may be supplied in an aqueous solution having a solids content ranging from about 10-60%, preferably 20-50%, more preferably 25-45% by weight of the polymer, with the remainder water. The composition of the acrylic copolymer may contain from about 0. 1-99 parts of the A monomer, and about 0.1-99 parts of the B monomer. Acrylic polymer solutions include those sold by Seppic, Inc., under the trade name Capigel.

Also suitable are acrylic polymeric thickeners that are copolymers of A, B, and C monomers wherein A and B are as defined above, and C has the general formula:

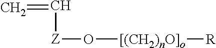

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the trade name Acrysol ICS-1.

Also suitable are acrylate-based anionic amphiphilic polymers containing at least one hydrophilic unit and at least one allyl ether unit containing a fatty chain. Preferred are those where the hydrophilic unit contains an ethylenically unsaturated anionic monomer, more specifically a vinyl carboxylic acid such as acrylic acid, methacrylic acid or mixtures thereof, and where the allyl ether unit containing a fatty chain corresponds to the monomer of the formula:

CH₂═CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C₁₈) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl(meth)acrylate, divinylbenzene, (poly)ethylene glycol dimethacrylate and methylenebisacrylamide. Commercial examples of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 allyl ether (40/50/10).

Also suitable are acrylate copolymers such as Polyacrylate-3 which is a copolymer of methacrylic acid, methylmethacrylate, methylstyrene isopropyl isocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C₁₀₋₃₀ alkyl acrylate crosspolymer which is a copolymer of C₁₀₋₃₀ alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames and have the CTFA name carbomer.

One particularly suitable type of aqueous phase thickening agent are acrylate-based polymeric thickeners sold by Clariant under the Aristoflex trademark such as Aristoflex AVC, which is ammonium acryloyldimethyltaurate/VP copolymer; Aristoflex AVL which is the same polymer as found in AVC dispersed in a mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.

### C. High Molecular Weight PEG or Polyglycerins

Also suitable as the aqueous phase thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CTFA names polyglycerin-20, polyglycerin-40, and the like.

In the event the compositions of the invention are in anhydrous or emulsion form, the composition will comprise an oil phase. Oily ingredients are desirable for the skin moisturizing and protective properties. Suitable oils include silicones, esters, vegetable oils, synthetic oils, including but not limited to those set forth herein. The oils may be volatile or nonvolatile, and are preferably in the form of a pourable liquid at room temperature. The term “volatile” means that the oil has a measurable vapor pressure or a vapor pressure of at least about 2 mm. of mercury at 20° C. The term “nonvolatile” means that the oil has a vapor pressure of less than about 2 mm. of mercury at 20° C. Suitable oils may include the following:

### A. Volatile Oils

Suitable volatile oils generally have a viscosity ranging from about 0.5 to 5 centistokes 25° C. and include linear silicones, cyclic silicones, paraffinic hydrocarbons, or mixtures thereof. Volatile oils may be used to promote more rapid drying of the skin care composition after it is applied to skin. Volatile oils are more desirable when the skin care products containing the P-type ATPase inhibitor compound or derivative thereof are being formulated for consumers that have combination or oily skin. The, term “combination” with respect to skin type means skin that is oily in some places on the face (such as the T-zone) and normal in others.

1. Volatile Silicones

Cyclic silicones are one type of volatile silicone that may be used in the composition. Such silicones have the general formula:

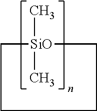

where n=3-6, preferably 4, 5, or 6.

Also suitable are linear volatile silicones, for example, those having the general formula:

(CH₃)₃Si—O—[Si(CH₃)₂—O]ₙ—Si(CH₃)₃

where n=0, 1, 2, 3, 4, or 5, preferably 0, 1, 2, 3, or 4.

Cyclic and linear volatile silicones are available from various commercial sources including Dow Corning Corporation and General Electric. The Dow Corning linear volatile silicones are sold under the tradenames Dow Corning 244, 245, 344, and 200 fluids. These fluids include hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof, with all viscosity measurements being at 25° C.

Suitable branched volatile silicones include alkyl trimethicones such as methyl trimethicone, a branched volatile silicone having the general formula:

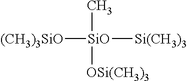

Methyl trimethicone may be purchased from Shin-Etsu Silicones under the tradename TMF-1.5, having a viscosity of 1.5 centistokes at 25° C.

2. Volatile Paraffinic Hydrocarbons

Also suitable as the volatile oils are various straight or branched chain paraffinic hydrocarbons having 5, 6, 7, 8, 9, 10, 11, 12, 13, 14, 15, 16, 17, 18, 19, or 20 carbon atoms, more preferably 8 to 16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference.

Preferred volatile paraffinic hydrocarbons have a molecular weight of 70-225, preferably 160 to 190 and a boiling point range of 30 to 320, preferably 60 to 260° C., and a viscosity of less than about 10 cst. at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable.

### B. Non-Volatile Oils

A variety of nonvolatile oils are also suitable for use in the compositions of the invention. The nonvolatile oils generally have a viscosity of greater than about 5 to 10 centistokes at 25° C., and may range in viscosity up to about 1,000,000 centipoise at 25° C. Examples of nonvolatile oils include, but are not limited to:

### 1. Esters

Suitable esters are mono-, di-, and triesters. The composition may comprise one or more esters selected from the group, or mixtures thereof.

(a) Monoesters

Monoesters are defined as esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, stearyl lactate, stearyl octanoate, stearyl stearate, isononyl isononanoate, and so on.

(b). Diesters

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

(c). Triesters

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂-₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition are further described in the C.T.F.A. Cosmetic Ingredient Dictionary and Handbook, Eleventh Edition, 2006, under the classification of “Esters”, the text of which is hereby incorporated by reference in its entirety.

### 2. Hydrocarbon Oils

It may be desirable to incorporate one or more nonvolatile hydrocarbon oils into the composition. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀-₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof. In one preferred embodiment such hydrocarbons have a molecular weight ranging from about 300 to 1000 Daltons.

### 3. Glyceryl Esters of Fatty Acids

Synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups. Glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

### 4. Nonvolatile Silicones

Nonvolatile silicone oils, both water soluble and water insoluble, are also suitable for use in the composition. Such silicones preferably have a viscosity ranging from about greater than 5 to 800,000 cst, preferably 20 to 200,000 cst at 25° C. Suitable water insoluble silicones include amine functional silicones such as amodimethicone.

For example, such nonvolatile silicones may have the following general formula:

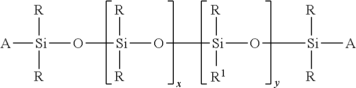

wherein R and R′ are each independently C₁₋₃₀ straight or branched chain, saturated or unsaturated alkyl, phenyl or aryl, trialkylsiloxy, and x and y are each independently 1-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁-₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Coming as Fluid 2502, or from DeGussa Care & Surface Specialties under the trade names Abil Wax 9801, or 9814.

### 5. Fluorinated Oils

Various types of fluorinated oils may also be suitable for use in the compositions including but not limited to fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers include those disclosed in U.S. Pat. Nos. 5,183,589, 4,803,067, 5,183,588, all of which are hereby incorporated by reference, which are commercially available from Montefluos under the trademark Fomblin.

In the case where the composition is anhydrous or in the form of an emulsion, it may be desirable to include one or more oil phase structuring agents in the cosmetic composition. The term “oil phase structuring agent” means an ingredient or combination of ingredients, soluble or dispersible in the oil phase, which will increase the viscosity, or structure, the oil phase. The oil phase structuring agent is compatible with the P-type ATPase inhibitor compound or derivative thereof, particularly if the P-type ATPase inhibitor compound or derivative thereof is soluble in the nonpolar oils forming the oil phase of the composition. The term “compatible” means that the oil phase structuring agent and P-type ATPase inhibitor compound or derivative thereof are capable of being formulated into a cosmetic product that is generally stable. The structuring agent may be present in an amount sufficient to provide a liquid composition with increased viscosity, a semi-solid, or in some cases a solid composition that may be self-supporting. The structuring agent itself may be present in the liquid, semi-solid, or solid form. Suggested ranges of structuring agent are from about 0.01 to 70%, preferably from about 0.05 to 50%, more preferably from about 0.1-35% by weight of the total composition. Suitable oil phase structuring agents include those that are silicone based or organic based. They may be polymers or non-polymers, synthetic, natural, or a combination of both. Such oil structuring agents may include the following:

### A. Silicone Structuring Agents

A variety of oil phase structuring agents may be silicone based, such as silicone elastomers, silicone gums, silicone waxes, and linear silicones having a degree of polymerization that provides the silicone with a degree of viscosity such that when incorporated into the cosmetic composition it is capable of increasing the viscosity of the oil phase. Examples of silicone structuring agents include, but are not limited to:

1. Silicone Elastomers

Silicone elastomers suitable for use in the compositions of the invention include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl)polysiloxanes, and dimethylvinylsiloxy-terminated dimethylsiloxane-methyl(3,3,-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Coming's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crosspolymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252; U.S. Pat. No. 5,760,116; U.S. Pat. No. 5,654,362; and Japanese Patent Application JP 61-18708; each of which is herein incorporated by reference in its entirety. It is particularly desirable to incorporate silicone elastomers into the compositions of the invention because they provide excellent “feel” to the composition, are very stable in cosmetic formulations, and relatively inexpensive.

2. Silicone Gums

Also suitable for use as an oil phase structuring agent are one or more silicone gums. The term “gum” means a silicone polymer having a degree of polymerization sufficient to provide a silicone having a gum-like texture. In certain cases the silicone polymer forming the gum may be crosslinked. The silicone gum typically has a viscosity ranging from about 500,000 to 100 million cst at 25° C., preferably from about 600,000 to 20 million, more preferably from about 600,000 to 12 million cst. All ranges mentioned herein include all subranges, e.g. 550,000; 925,000; 3.5 million.

The silicone gums that are used in the compositions include, but are not limited to, those of the general formula:

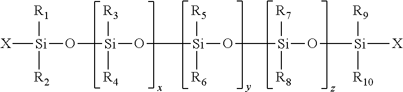

wherein R₁ to R₉ are each independently an alkyl having 1 to 30 carbon atoms, aryl, or aralkyl; and X is OH or a C₁-₃₀ alkyl, or vinyl; and wherein x, y, or z may be zero with the proviso that no more than two of x, y, or z are zero at any one time, and further that x, y, and z are such that the silicone gum has a viscosity of at least about 500,000 cst, ranging up to about 100 million centistokes at 25° C. Preferred is where R is methyl or OH.

Such silicone gums may be purchased in pure form from a variety of silicone manufacturers including Wacker-Chemie or Dow Corning, and the like. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1401. The silicone gum may also be purchased in the form of a solution or dispersion in a silicone compatible vehicle such as volatile or nonvolatile silicone. An example of such a mixture may be purchased from Barnet Silicones under the HL-88 tradename, having the INCI name dimethicone.

3. Silicone Waxes

Another type of oily phase structuring agent includes silicone waxes that are typically referred to as alkyl silicone waxes which are semi-solids or solids at room temperature. The term “alkyl silicone wax” means a polydimethylsiloxane having a substituted long chain alkyl (such as C16 to 30) that confers a semi-solid or solid property to the siloxane. Examples of such silicone waxes include stearyl dimethicone, which may be purchased from DeGussa Care & Surface Specialties under the tradename Abil Wax 9800 or from Dow Corning under the tradename 2503. Another example is bis-stearyl dimethicone, which may be purchased from Gransil Industries under the tradename Gransil A-18, or behenyl dimethicone, behenoxy dimethicone.

4. Polyamides or Silicone Polyamides

Also suitable as oil phase structuring agents are various types of polymeric compounds such as polyamides or silicone polyamides.

The term silicone polyamide means a polymer comprised of silicone monomers and monomers containing amide groups as further described herein. The silicone polyamide preferably comprises moieties of the general formula:

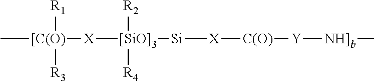

X is a linear or branched alkylene having from about 1-30 carbon atoms; R₁, R₂, R₃, and R₄ are each independently C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

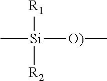

### and Y is:

(a) a linear or branched alkylene having from about 1-40 carbon atoms which may be substituted with:

(i) one or more amide groups having the general formula R₁CONR₁, or

(ii) C₅₋₆ cyclic ring, or

(iii) phenylene which may be substituted with one or more C₁₋₁₀ alkyl groups, or

(iv) hydroxy, or

(v) C₃₋₈ cycloalkane, or

(vi) C₁₋₂₀ alkyl which may be substituted with one or more hydroxy groups, or

(vii) C₁₋₁₀ alkyl amines; or
(b) TR₅R₆R₇
   wherein R₅, R₆, and R₇, are each independently a C₁₋₁₀ linear or branched alkylenes, and T is CR₈ wherein R₈ is hydrogen, a trivalent atom N, P, or Al, or a C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

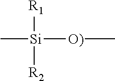

Preferred is where R₁, R₂, R₃, and R₄ are C₁₋₁₀, preferably methyl; and X and Y are a linear or branched alkylene. Preferred are silicone polyamides having the general formula:

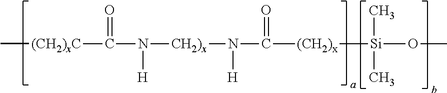

wherein a and b are each independently sufficient to provide a silicone polyamide polymer having a melting point ranging from about 60 to 120° C., and a molecular weight ranging from about 40;000 to 500,000 Daltons. One type of silicone polyamide that may be used in the compositions of the invention may be purchased from Dow Corning Corporation under the tradename Dow Corning 2-8178 gellant which has the CTFA name nylon-611/dimethicone copolymer which is sold in a composition containing PPG-3 myristyl ether.

Also suitable are polyamides such as those purchased from Arizona Chemical under the tradenames Uniclear and Sylvaclear. Such polyamides may be ester terminated or amide terminated. Examples of ester terminated polyamides include, but are not limited to those having the general formula:

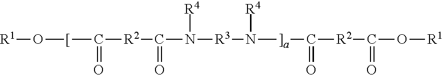

wherein n denotes a number of amide units such that the number of ester groups ranges from about 10% to 50% of the total number of ester and amide groups; each R₁ is independently an alkyl or alkenyl group containing at least 4 carbon atoms; each R₂ is independently a C₄₋₄₂ hydrocarbon group, with the proviso that at least 50% of the R₂ groups are a C₃₀₋₄₂ hydrocarbon; each R₃ is independently an organic group containing at least 2 carbon atoms, hydrogen atoms and optionally one or more oxygen or nitrogen atoms; and each R₄ is independently a hydrogen atom, a C₁₋₁₀ alkyl group or a direct bond to R₃ or to another R₄, such that the nitrogen atom to which R₃ and R₄ are both attached forms part of a heterocyclic structure defined by R₄—N—R₃, with at least 50% of the groups R₄ representing a hydrogen atom.

General examples of ester and amide terminated polyamides that may be used as oil phase gelling agents include those sold by Arizona Chemical under the tradenames Sylvaclear A200V or A2614V, both having the CTFA name ethylenediamine/hydrogenated dimer dilinoleate copolymer/bis-di-C₁₄₋₁₈ alkyl amide; Sylvaclear AF1900V; Sylvaclear C75V having the CTFA name bis-stearyl ethylenediamine/neopentyl glycol/stearyl hydrogenated dimer dilinoleate copolymer; Sylvaclear PA1200V having the CTFA name Polyamide-3; Sylvaclear PE400V; Sylvaclear WF1500V; or Uniclear, such as Uniclear 100VG having the INCI name ethylenediamine/stearyl dimer dilinoleate copolymer; or ethylenediamine/stearyl dimer ditallate copolymer. Other examples of suitable polyamides include those sold by Henkel under the Versamid trademark (such as Versamid 930, 744, 1655), or by Olin Mathieson Chemical Corp. under the brand name Onamid S or Onamid C.

5. Natural or Synthetic Organic Waxes

Also suitable as the oil phase structuring agent may be one or more natural or synthetic waxes such as animal, vegetable, or mineral waxes. Preferably such waxes will have a higher melting point such as from about 50 to 150° C., more preferably from about 65 to 100° C. Examples of such waxes include waxes made by Fischer-Tropsch synthesis, such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, and so on.

6. Montmorillonite Minerals

One type of structuring agent that may be used in the composition comprises natural or synthetic montmorillonite minerals such as hectorite, bentonite, and quaternized derivatives thereof, which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like.

7. Silicas and Silicates

Another type of structuring agent that may be used in the compositions are silicas, silicates, silica silylate, and alkali metal or alkaline earth metal derivatives thereof. These silicas and silicates are generally found in the particulate form and include silica, silica silylate, magnesium aluminum silicate, and the like.

The composition may contain one or more surfactants, especially if in the emulsion form. However, such surfactants may be used if the compositions are anhydrous also, and will assist in dispersing ingredients that have polarity, for example pigments. Such surfactants may be silicone or organic based. The surfactants will aid in the formation of stable emulsions of either the water-in-oil or oil-in-water form. If present, the surfactant may range from about 0.001 to 30%, preferably from about 0.005 to 25%, more preferably from about 0.1 to 20% by weight of the total composition.

### A. Silicone Surfactants

Suitable silicone surfactants include polyorganosiloxane polymers that have amphiphilic properties, for example contain hydrophilic radicals and lipophilic radicals. These silicone surfactants may be liquids or solids at room temperature.

1. Dimethicone Copolyols or Alkyl Dimethicone Copolyols

One type of silicone surfactant that may be used is generally referred to as dimethicone copolyol or alkyl dimethicone copolyol. This surfactant is either a water-in-oil or oil-in-water surfactant having an Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One type of suitable silicone surfactant has the general formula:

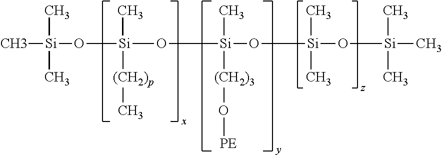

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the trade name Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Coming 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

2. Crosslinked Silicone Surfactants

Also suitable are various types of crosslinked silicone surfactants that are often referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986, U.S. Pat. No. 5,412,004, U.S. Pat. No. 5,837,793 and U.S. Pat. No. 5,811,487, the contents of which are hereby incorporated by reference in their entireties.

Polyoxyalkylenated silicone elastomers that may be used in at least one embodiment of the invention include those sold by Shin-Etsu Silicones under the names KSG-21 , KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

One preferred crosslinked silicone elastomer emulsifier is dimethicone/PEG-10/15 crosspolymer, which provides excellent aesthetics due to its elastomeric backbone, but also surfactancy properties.

### B. Organic Nonionic Surfactants

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanoates such as PEG dipolyhydroxystearate wherein the number of repeating ethylene glycol units ranges from 3 to 1000.

Also suitable as nonionic surfactants are those formed by the reaction of a carboxylic acid with an alkylene oxide or with a polymeric ether. The resulting products have the general formula:

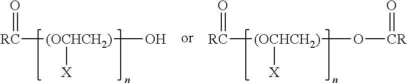

where RCO is the carboxylic ester radical, X is hydrogen or lower alkyl, and n is the number of polymerized alkoxy groups. In the case of the diesters, the two RCO-groups do not need to be identical. Preferably, R is a C6-30 straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula:

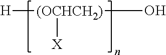

wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

Certain types of amphoteric, zwitterionic, or cationic surfactants may also be used in the compositions. Descriptions of such surfactants are set forth in U.S. Pat. No. 5,843,193, which is hereby incorporated by reference in its entirety.

It may be desirable to include one or more penetration enhancers in the composition. Penetration enhancers are ingredients that enhance the penetration of the Type I H⁺, K⁺-ATPase inhibitor compound or derivative thereof into the keratinous surface to which the composition is applied. If present, suitable penetration enhancers may range from about 0.001 to 30%, preferably from about 0.005 to 25%, more preferably from about 0.01 to 20%. Suitable penetration enhancers include, but are not limited to, lipophilic materials such as saturated or unsaturated C₆₋₄₀ straight or branched chain fatty acids, or saturated or unsaturated C₆₋₄₀ straight or branched chain fatty alcohols. Examples include oleic acid, linoleic acid, stearic acid, oleyl alcohol, linoleyl alcohol, and the like.

It may also be desirable to include one or more humectants in the composition. If present, such humectants may range from about 0.001 to 25%, preferably from about 0.005 to 20%, more preferably from about 0.1 to 15% by weight of the total composition. Examples of suitable humectants include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄ alkylene glycols, most particularly butylene glycol.

It may be desirable to include one or more botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including yeast ferment extract, Padina pavonica extract, Thermus thermophilis ferment extract, Camelina sativa seed oil, Boswellia serrata extract, olive extract, Aribodopsis thaliana extract, Acacia dealbata extract, Acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza glabra, Salix nigra, Macrocycstis pyrifera, Pyrus malus, Saxifraga sarmentosa, Vitis vinifera, Morus nigra, Scutellaria baicalensis, Anthemis nobilis, Salvia sclarea, Rosmarinus officianalis, Citrus medica Limonum, Panax, Ginseng, Siegesbeckia orientalis, Fructus mume, Ascophyllum nodosum, Bifida Ferment lysate, Glycine soja extract, Beta vulgaris, Haberlea rhodopensis, Polygonum cuspidatum, Citrus Aurantium dulcis, Vitis vinifera, Selaginella tamariscina, Humulus lupulus, Citrus reticulata Peel, Punica granatum, Asparagopsis, Curcuma longa, Menyanthes trifoliata, Helianthus annuus, Hordeum vulgare, Cucumis sativus, Evernia prunastri, Evernia furfuracea, and mixtures thereof.

It may be desirable to include one or more tyrosinase inhibiting agents in the compositions of the invention. Such tyrosinase inhibitors may include, but are not limited to, kojic acid, arbutin and hydroquinone.

It may be desirable to include one or more additional skin-lightening compounds in the compositions of the present invention. Suitable skin-lightening compounds include, but are not limited to, ascorbic acid and its derivatives, e.g., magnesium ascorbyl phosphate, ascorbyl glucosamine, ascorbyl palmitate. Other skin-lightening agents include adapalene, aloe extract, ammonium lactate, anethole derivatives, apple extract, azelaic acid, bamboo extract, bearberry extract, bletilla tuber, Bupleurum falcatum extract, burnet extract, butyl hydroxy anisole, butyl hydroxy toluene, deoxyarbutin, 1,3 diphenyl propane derivatives, 2,5 dihydroxybenzoic acid and its derivatives, 2-(4-acetoxyphenyl)-1,3 dithane, 2-(4-hydroxyphenyl)-1,3 dithane, ellagic acid, escinol, estragole derivatives, FADE OUT (available from Pentapharm), Fangfeng, fennel extract, ganoderma extract, gaoben, GATULINE WHITENING (available from Gattlefosse), genistic acid and its derivatives, glabridin and its derivatives, gluco pyranosyl-1-ascorbate, gluconic acid, glycolic acid, green tea extract, placenta extract, 4-Hydroxy-5-methyl-3[2H]-furanone, 4 hydroxyanisole and its derivatives, 4-hydroxy benzoic acid derivatives, hydroxycaprylic acid, inositol ascorbate, lactic acid, lemon extract, linoleic acid, MELA WHITE (available from Pentapharm), Morus alba extract, mulberry root extract, niacinamide, 5-octanoyl salicylic acid, parsley extract, phellinus linteus extract, pyrogallol derivatives, retinoic acid, retinol, retinyl esters (acetate, propionate, palmitate, linoleate), 2,4 resorcinol derivatives, 3,5 resorcinol derivatives, rose fruit extract, salicylic acid, 3,4,5 trihydroxybenzyl derivatives, tranexamic acid, vitamin D3 and its analogs, and mixtures thereof.

It may also be desirable to include one or more sunscreens in the compositions of the invention. Such sunscreens include chemical UVA or UVB sunscreens or physical sunscreens in the particulate form. Inclusion of sunscreens in the compositions containing the P-type ATPase inhibitor compound or derivative thereof will provide additional protection to skin during daylight hours and promote the effectiveness of the P-type ATPase inhibitor compound or derivative thereof on the skin. Such sunscreen compounds may include the following:

### A. UVA Chemical Sunscreens

If desired, the composition may comprise one or more UVA sunscreens. The term “UVA sunscreen” means a chemical compound that blocks UV radiation in the wavelength range of about 320 to 400 nm. Preferred UVA sunscreens are dibenzoylmethane compounds having the general formula:

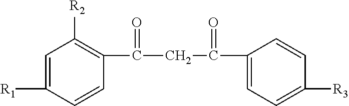

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

Other types of UVA sunscreens include dicamphor sulfonic acid derivatives, such as ecamsule, a sunscreen sold under the trade name Mexoryl™, which is terephthalylidene dicamphor sulfonic acid, having the formula:

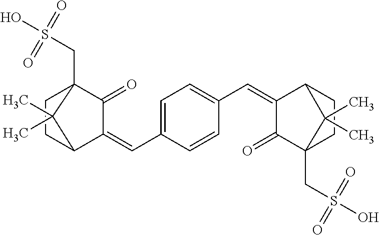 [SMILES: CC1(C)C2CCC1(CS(=O)(=O)O)C(=O)/C2=C\c1ccc(/C=C2\C(=O)C3(CS(=O)(=O)O)CCC2C3(C)C)cc1]

The composition may contain from about 0.001-20%, preferably 0.005-5%, more preferably about 0.005-3% by weight of the composition of UVA sunscreen. In the preferred embodiment of the invention the UVA sunscreen is Avobenzone, and it is present at not greater than about 3% by weight of the total composition.

### B. UVB Chemical Sunscreens

The term “UVB sunscreen” means a compound that blocks UV radiation in the wavelength range of from about 290 to 320 nm. A variety of UVB chemical sunscreens exist including alpha-cyano-beta,beta-diphenyl acrylic acid esters as set forth in U.S. Pat. No. 3,215,724, which is hereby incorporated by reference in its entirety. One particular example of an alpha-cyano-beta,beta-diphenyl acrylic acid ester is Octocrylene, which is 2-ethylhexyl 2-cyano-3,3-diphenylacrylate. In certain cases the composition may contain no more than about 110% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

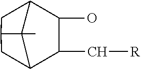

wherein R is p-tolyl or styryl, preferably styryl. Particularly preferred is 4-methylbenzylidene camphor, which is a lipid soluble UVB sunscreen compound sold under the tradename Eusolex 6300 by Merck. Also suitable are cinnamate derivatives having the general formula:

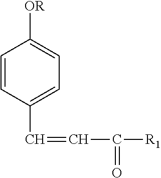

wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as UVB screening agents are various benzophenone derivatives having the general formula:

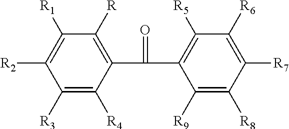

wherein R through R₉ are each independently H, OH, NaO₃S, SO₃H, SO₃Na, Cl, R″, OR″ where R″ is C₁₋₂₀ straight or branched chain alkyl Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone), Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

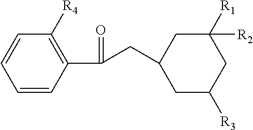

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

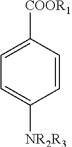

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula:

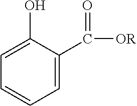

wherein R is a straight or branched chain alkyl, including derivatives of the above compound formed from mono-, di-, or triethanolamines. Particular preferred are octyl salicylate, TEA-salicylate, DEA-salicylate, and mixtures thereof.

Generally, the amount of the UVB chemical sunscreen present may range from about 0.001-45%, preferably 0.005-40%, more preferably about 0.01-35% by weight of the total composition.

If desired, the compositions of the invention may be formulated to have a certain SPF (sun protective factor) values ranging from about 1-50, preferably about 2-45, most preferably about 5-30. Calculation of SPF values is well known in the art.

The compositions of the invention may contain particulate materials in the form of pigments, inert particulates, or mixtures thereof. If present, suggested ranges are from about 0.01-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition. Suitable particulate materials may include the following:

### A. Powders

The particulate matter may be colored or non-colored (for example white) non-pigmented powders. Suitable non-pigmented powders include, but are not limited to, bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

### B. Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

The composition may contain 0.001-8%, preferably 0.01-6%, more preferably 0.05-5% by weight of the total composition of preservatives. A variety of preservatives are suitable, including, but not limited to, benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, caprylyl glycol, biguanide derivatives, phenoxyethanol, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and the like. In one preferred embodiment the composition is free of parabens.

The compositions of the invention may contain vitamins and/or coenzymes, as well as antioxidants. If so, 0.001-10%, preferably 0.01-8%, more preferably 0.05-5% by weight of the total composition is suggested. Suitable vitamins include ascorbic acid and derivatives thereof such as ascorbyl palmitate, tetrahexydecyl ascorbate, and so forth; the B vitamins such as thiamine, riboflavin, pyridoxin, niacin, niacinamide, nicotinic acid, nicotinic acid dinucleotide, and so forth, as well as coenzymes such as thiamine pyrophoshate, flavin adenine dinucleotide, folic acid, pyridoxal phosphate, tetrahydrofolic acid, and so forth. Also Vitamin A and derivatives thereof are suitable. Examples are retinyl palmitate, retinol, retinoic acid, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so forth.

It may be desirable to include one or more film forming ingredients in the cosmetic compositions of the invention. Suitable film formers are ingredients that contribute to formation of a film on the keratinous surface. In some cases the film formers may provide films that provide long wearing or transfer resistant properties such that the cosmetic applied to the keratinous surface will remain for periods of time ranging from 3 to 16 hours. If present, such film formers may range from about 0.01 to 50%, preferably from about 0.1 to 40%, more preferably from about 0.5 to 35% by weight of the total composition. The film formers are most often found in the polymeric form and may be natural or synthetic polymers. If synthetic, silicone polymers, organic polymers or copolymers of silicones and organic groups may be acceptable. Suitable film formers include, but are not limited to:

### A. Silicone Resins

One particularly suitable type of silicone film former is a silicone resin. Silicone resins are generally highly crosslinked structures comprising combinations of M, D, T, and Q units. The term “M” means a monofunctional siloxy unit having the general formula:

[Si—(CH₃)₃—O]_{0.5}

In cases where the M unit is other than methyl (such as ethyl, propyl, ethoxy, etc.) the M unit may have a prime after it, e.g. M′.

The term “D” means a difunctional siloxy unit having the general formula:

Si—(CH₃)₂—O]_{1.0}
The difunctional unit may be substituted with alkyl groups other than methyl, such as ethyl, propyl, alkylene glycol, and the like, in which case the D unit may be referred to as D′, with the prime indicating a substitution.

The term “T” means a trifunctional siloxy unit having the general formula:

[Si—(CH₃)—O]_{1.5}

The trifunctional unit may be substituted with substituents other than methyl, in which case it may be referred to as T′.

The term “Q” refers to a quadrifunctional siloxy unit having the general formula:

[Si—O—]_{2.0}

The silicone resins that may be used as film formers in the compositions of the invention preferably comprise highly crosslinked combinations of M, T, and Q units. Examples of such resins include trimethylsiloxysilicate which can be purchased from Dow Corning Corporation as 749 Fluid, or from GE Silicones under the SR-1000 tradename. Also suitable is a silicone resin that contains a large percentage of T groups, such as MK resin sold by Wacker-Chemie, having the CTFA name polymethylsilsesquioxane.

### B. Copolymers of Silicone and Organic Monomers

Also suitable for use as the film formers are copolymers of silicone and organic monomers such as acrylates, methacrylates, and the like. Examples of such suitable film forming polymers include those commonly referred to as silicone acrylate or vinyl silicone copolymers, such as those sold by 3M under the brand name “Silicone Plus” polymers such as SA-70, having the CTFA name Polysilicone-7 and is a copolymer of isobutylmethacrylate and n-butyl endblocked polydimethylsiloxane propyl methacrylate; or VS-70 having the CTFA name Polysilicone-6, which is a copolymer of dimethylsiloxane and methyl-3 mercaptopropyl siloxane reacted with isobutyl methacrylate; or VS-80, having the CTFA name Polysilicone-8, which has the general structure:

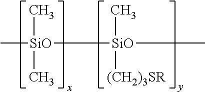

where R represents the acrylates copolymer radical.

### C. Organic Polymers

Also suitable as film formers include various types of organic polymers such as polymers formed from acrylic acid, methacrylic acid, or their simple C₁₋₁₀ carboxylic acid esters, such as methyl methacrylate, methyl acrylate, and the like.

Also suitable are various types of natural polymers such as shellac, natural resins, chitin, and the like.

It may also be desirable to incorporate one or more DNA repair enzymes into the composition of the invention. Suggested ranges are from about 0.00001 to about 35%, preferably from about 0.00005 to about 30%, more preferably from about 0.0001 to about 25% of one or more DNA repair enzymes.

DNA repair enzymes as disclosed in U.S. Pat. Nos. 5,077,211; 5,190,762; 5,272,079; and 5,296,231, all of which are hereby incorporated by reference in their entirety, are suitable for use in the compositions and method of the invention. One example of such a DNA repair enzyme may be purchased from AGI Dermatics under the trade name Roxisomes®, and has the INCI name Arabidopsis Thaliana extract. It may be present alone or in admixture with lecithin and water. This DNA repair enzyme is known to be effective in repairing 8-oxo-diGuanine base mutation damage.

Another type of DNA repair enzyme that may be used is one that is known to be effective in repairing 06-methyl guanine base mutation damage. It is sold by AGI Dermatics under the trade name Adasomes®, and has the INCI name Lactobacillus ferment, which may be added to the composition of the invention by itself or in admixture with lecithin and water.

Another type of DNA repair enzyme that may be used is one that is known to be effective in repairing T-T dimers. The enzymes are present in mixtures of biological or botanical materials. Examples of such ingredients are sold by AGI Dermatics under the trade names Ultrasomes® or Photosomes®. Ultrasomes® comprises a mixture of Micrococcus lysate (an end product of the controlled lysis of a species of micrococcus), lecithin, and water. Photosomes® comprises a mixture of plankton extract (which is the extract of a biomass which includes enzymes from one or more of the following organisms: thalassoplankton, green micro-algae, diatoms, greenish-blue and nitrogen-fixing seaweed), water, and lecithin.

Another type of DNA repair enzyme may be a component of various inactivated bacterial lysates such as Bifida lysate or Bifida ferment lysate, the latter a lysate from Bifido bacteria which contains the metabolic products and cytoplasmic fractions when Bifido bacteria are cultured, inactivated and then disintegrated. This material has the INCI name Bifida Ferment Lysate.

Other suitable DNA repair enzymes include Endonuclease V, which may be produced by the denV gene of the bacteriophage T4. Also suitable are T4 endonuclease; O-6-methylguanine-DNA methyltransferases; photolyases, base glycosylases such as uracil- and hypoxanthine-DNA glycosylases; apyrimidinic/apurinic endonucleases; DNA exonucleases, damaged-bases glycosylases (e.g., 3-methyladenine-DNA glycosylase); correndonucleases either alone or in complexes (e.g., E. coli uvrA/uvrB/uvrC endonuclease complex); APEX nuclease, which is a multi-functional DNA repair enzyme often referred to as “APE”; dihydrofolate reductase; terminal transferase; polymerases; ligases; and topoisomerases.

Other types of suitable DNA repair enzymes may be categorized by the type of repair facilitated and include BER (base excision repair) or BER factor enzymes such as uracil-DNA glycosylase (UNG); single strand selective monofunctional uracil DNA glycosylase (SMUG1); 3,N(4)-ethenocytosine glycosylase (MBD4); thymine DNA-glycosylase (TDG); A/G-specific adenine DNA glycosylase (MUTYH); 8-oxoguanine DNA glycosylase (OGG1); endonuclease III-like (NTHL1); 3-methyladenine DNA glycosidase (MPG); DNA glycosylase/AP lyase (NEIL1 or 2); AP endonuclease (APEX 1 and 2), DNA ligase (LIG3), ligase accessory factor (XRCC1); DNA 5′-kinase/3′-phosphatase (PNKP); ADP-ribosyltransferase (PARP1 or 2).

Another category of DNA repair enzymes includes those that are believed to directly reverse damage such as O-6-MeG alkyl transferase (MGMT); 1-meA dioxygenase (ALKBH2 or ALKBH3).

Yet another category of enzymes operable to repair DNA/protein crosslinks includes Tyr-DNA phosphodiesterase (TDP1).

Also suitable are MMR (mismatch excision repair) DNA repair enzymes such as MutS protein homolog (MSH2); mismatch repair protein (MSH3); mutS homolog 4 (MSH4); MutS homolog 5 (MSH5); or G/T mismatch-binding protein (MSH6); DNA mismatch repair protein (PMS1, PMS2, MLH1, MLH3); Postmeiotic segregation increased 2-like protein (PMS2L3); or postmeiotic segregation increased 2-like 4 pseudogene (PMS2L4).

Also suitable are DNA repair enzymes are those known as nucleotide excision repair (NER) enzymes and include those such as Xeroderma Pigmentosum group C-complementing protein (XPC); RAD23 (S. cerevisiae) homolog (RAD23B); caltractin isoform (CETN2); RFA Protein 1, 2, of 3 (RPA1, 2, or 3); 3′ to 5′ DNA helicase (ERCC3); 5′ to 3′ DNA helicase (ERCC2); basic transcription factor (GTF2H1, GTF2H2, GTF2H3, GTF2H4, GTF2H5); CDK activating kinase (CDK7, CCNH); cyclin G1-interacting protein (MNAT1); DNA excision repair protein ERCC-1 or RAD-51; excision repair cross-complementing 1 (ERCC1); DNA ligase 1 (LIG1); ATP-dependent helicase (ERCC6); and the like.

Also suitable may be DNA repair enzymes in the category that facilitate homologous recombination and include, but are not limited to DNA repair protein RAD51 homolog (RAD51, RAD51L1, RAD51B etc.); DNA repair protein XRCC2; DNA repair protein XRCC3; DNA repair protein RAD52; ATPase (RAD50); 3′ exonuclease (MRE11A); and so on.

DNA repair enzymes that are DNA polymerases are also suitable and include DNA polymerase beta subunit (POLB); DNA polymerase gamma (POLG); DNA polymerase subunit delta (POLD1); DNA polymerase II subunit A (POLE); DNA polymerase delta auxiliary protein (PCNA); DNA polymerase zeta (POLZ); MAD2 homolog (REV7); DNA polymerase eta (POLH): DNA polymerase kappa (POLK): and the like.

Various types of DNA repair enzymes that are often referred to as “editing and processing nucleases” include 3′-nuclease; 3′-exonuclease; 5′-exonuclease; endonuclease; and the like.

Other examples of DNA repair enzymes include DNA helicases, such as ATP DNA helicase, and so forth.

The DNA repair enzymes may be present as components of botanical extracts, bacterial lysates, biological materials, and the like. For example, botanical extracts may contain DNA repair enzymes.

In accordance with a third aspect of the present invention, there is provided a method of inhibiting ATP7A and/or ATP7B, other than by affecting a V—H+-ATPase, in a subject, the method comprising administering to the subject in need thereof a composition containing, comprising or consisting essentially of, a small molecule inhibitor of ATP7A and/or ATP7B having the formula I or the formula II in a cosmetically-, dermatologically- or pharmaceutically-acceptable vehicle, carrier or diluent therefor, as described hereinabove.

In accordance with a fourth aspect of the present invention, there is provided a method of inhibiting melanogenesis in skin cells of a subject, other than by affecting a V—H+-ATPase, the method comprising administering to the subject in need thereof a composition containing, comprising or consisting essentially of, a small molecule inhibitor of ATP7A having the formula I or the formula II in a cosmetically-, dermatologically- or pharmaceutically-acceptable vehicle, carrier or diluent therefor as described hereinabove. In some preferred embodiments of this aspect of the present invention, the small molecule inhibitor has the formula:

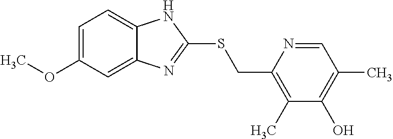

or the formula:

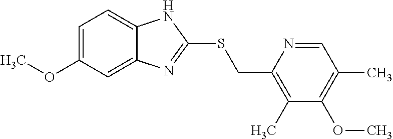

In accordance with a fifth aspect of the present invention, there is provided a method of inhibiting the level of copper ions in cells, other than by a method of chelating or binding the copper ions, the method comprising treating the cells with a composition containing, comprising or consisting essentially of, a small molecule inhibitor of ATP7A or ATP7B, having the formula I or the formula II. The small molecule inhibitor can be formulated in a cosmetically-, dermatologically- or pharmaceutically-acceptable vehicle, carrier or diluent therefore, consistent with its chemical stability, as described hereinabove. The composition containing the small molecule inhibitor should be applied as locally as possible to minimize the systemic exposure, such as near the site of the cells creating excessive copper or similar ion concentrations. It may be applied for a short period to ameliorate symptoms of excessive copper or similar ions in cells, or administered for a long time for congenital or genetic diseases related to excess copper or related ions. It may be used in conjunction with (in the same formulation, at the same time, and/or in the same temporal regimen as) other cosmetic, dermatological or pharmaceutical ingredients that enhance their activity or ameliorate other symptoms of the excessive copper or related ions.

In accordance with a sixth aspect of the present invention, there is provided a method of prophylaxis or treatment of Alzheimer's disease, by reversing up-regulation of ATP7A and thereby reducing excretion of copper ions by ATP7A in cells of a subject, other than by affecting a V—H+-ATPase, comprising the step of administering to the subject in need thereof a composition containing, comprising or consisting essentially of, a compound of the formula I or the formula II in a cosmetically-, dermatologically- or pharmaceutically-acceptable vehicle, carrier or diluent therefor.

In accordance with a seventh aspect of the present invention, there is provided a method of overcoming anti-cancer drug-, e.g., cisplatin-, resistance in tumor cells of a subject, other than by affecting a V—H+-ATPase, and preferably without increasing pigmentation in the skin cells of the subject, the method comprising administering to the subject in need thereof, a composition comprising a small molecule inhibitor of ATP7A and/or ATP7B having the formula I or the formula II in a cosmetically-, dermatologically- or pharmaceutically-acceptable vehicle, carrier or diluent therefor.

The invention is further illustrated by the following non-limiting examples.

### EXAMPLES

### Example I

### Omeprazole Inhibition of Cu⁺²-Stimulated ATP7A Trafficking

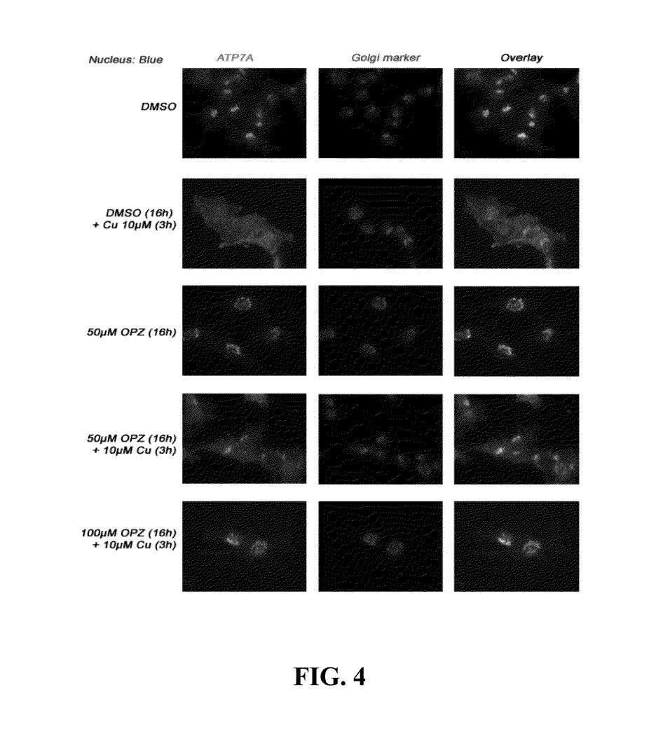

Cells were incubated for 16 hours with 100 μM omeprazole or the vehicle (DMSO) and then treated with Cu+2, or not treated, to determine if omeprazole could inhibit Cu+2-stimulated (CuCl₂) ATP7A trafficking. It was found that short incubation periods with omeprazole of about 1-2 hours were not sufficient to block Cu+²-stimulated ATP7A trafficking/relocalization from the perinuclear Golgi to the plasma membrane (data not shown). However, as indicated in the images of co-localization assays, using fluorescent markers for Golgi complex and ATP7A, showing Cu+²-stimulated ATP7A trafficking, in FIG. 4, a robust inhibition of this re-localization occurs upon treatment of cells with omeprazole for 16 hours. ATP7A is seen as green following immunofluorescence protocols previously described (Petris et al., 1999), the Golgi stained red, and the nucleus stained blue. The top row of images indicates that under basal conditions, ATP7A is localized in the perinuclear region of the Golgi complex. The second row of images indicates that stimulation of cells with 10 μM Cu⁺² initiates trafficking of ATP7A away from the Golgi. In the third row of images, it is observed that incubation of cells in 10 μM omeprazole, in the absence of Cu⁺²-stimulation, does not modify ATP7A from baseline. The fourth row of images shows that incubating cells for 16 hours in 50 μM omeprazole, followed by a 3 hour incubation of cells in 10 μM Cu⁺², results in a reduction in ATP7A trafficking away from the Golgi. From the fifth row of images, it can be seen that incubating cells in 100 μM omeprazole for 16 hours, followed by a 3 hour incubation in 10 μM Cu⁺², also effectively inhibits ATP7A trafficking, as the ATP7A is seen to be tightly localized in the Golgi. These results suggested to the inventors that, using a copper-stimulation model, omeprazole inhibits ATP7A trafficking but may require time to undergo activation by metabolism or re-arrangement, or may require time to reach the site of action.

### Example II

### Pre-Treatment of Cells Containing Tyrosinase and ATP7A with Omeprazole Reduces Melanin Content

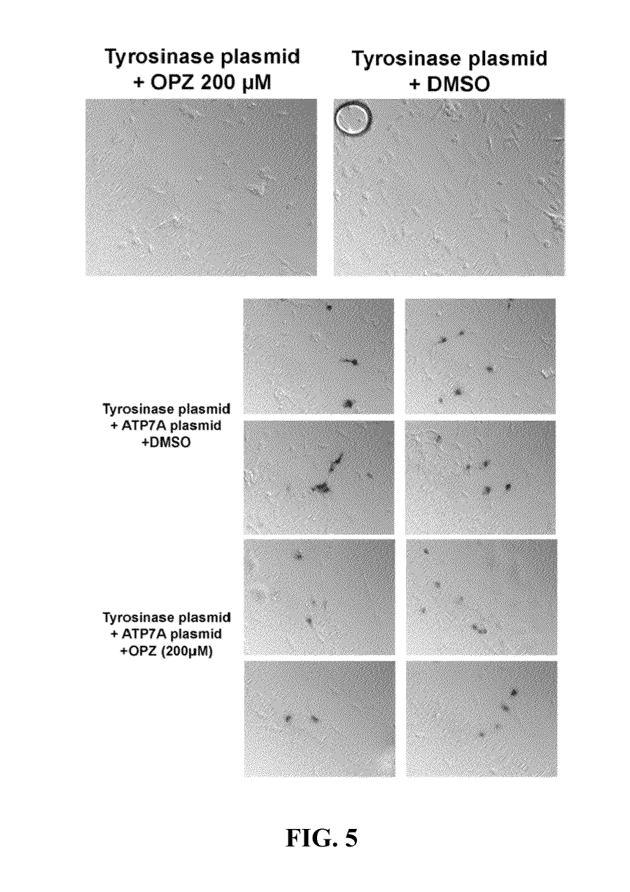

FIG. 5 summarizes experiments performed in Menkes patient Me32a fibroblasts which genetically lack functional ATP7A. Therefore, in order to synthesize melanin, cells must be transfected with both tyrosinase and ATP7A. In this experiment, cells were transfected with a tyrosinase expression plasmid alone or in combination with an ATP7A plasmid using Lipofectamine. Menkes patient fibroblasts (lacking ATP7A) were pretreated with omeprazole or DMSO overnight prior to transfection with a tyrosinase expression plasmid alone or in combination with ATP7A plasmid. Cells were incubated for an additional 24 hours to allow plasmid expression in continued presence of DMSO or omeprazole. Tyrosinase activity was then assayed in situ by fixing cells on the culture dish, incubating with L-DOPA and assaying for the formation of the brown pigment, DOPA-chrome. The images in FIG. 5, representing light microscopic photographs of in situ tyrosinase activity in Me32a Menkes disease fibroblasts, indicate that, under conditions in which the cells contain no ATP7A, there is no melanin synthesized (first row). Cells containing both tyrosinase and ATP7A are observed to contain melanin (second and third rows). Diminished melanin content is indicated in cells containing both tyrosinase and ATP7A but pretreated with 200 μM omeprazole (rows 4 and 5). The results strongly suggested that omeprazole lowers ATP7A-dependent tyrosinase activity.

### Example III

### SCH-28080 Inhibits Cu+²-Stimulated ATP7A Re-Localization

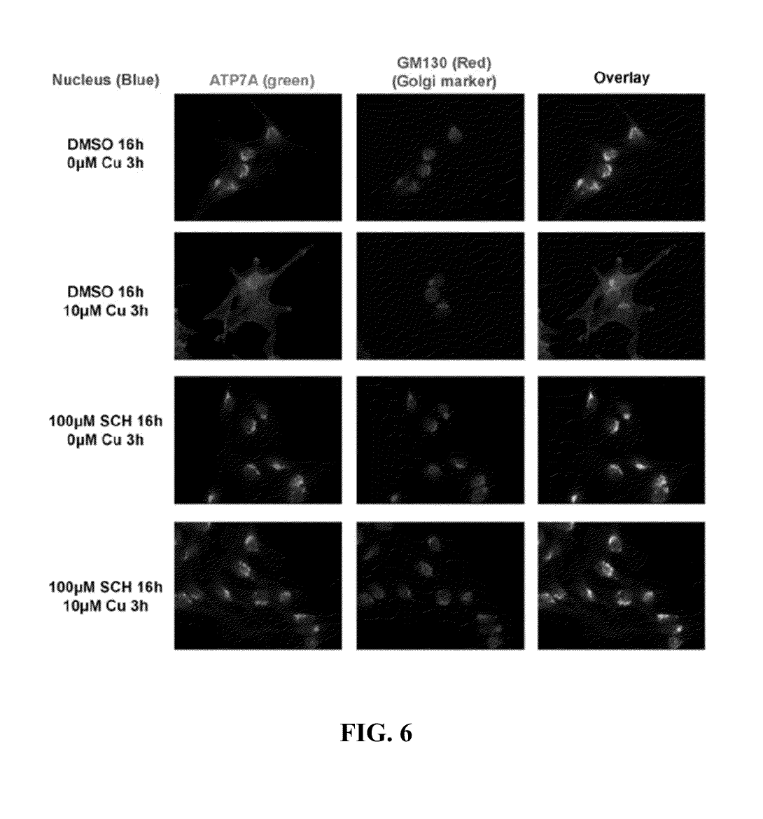

The inventors next sought to ascertain whether other P-type I H+, K+-ATPases, which are structurally and functionally related to substituted benzimidazoles, also interfere with ATP7A trafficking. Using a co-localization assay, in the copper-stimulation model, used with omeprazole, as described above, B16 cells were pretreated overnight (for 16 hours) with DMSO or 100 μM of the substituted-imidazopyridine compound, SCH-28080, followed by a 3 hour incubation with 10 μM Cu+² to elicit ATP7A trafficking. ATP7A and the Golgi marker GM130 were detected by immunofluorescence microscopy using affinity purified ATP7A antibodies, and Alexa488 anti-rabbit secondary antibodies (available from Molecular Probes, Carlsbad, Calif.). The ATP7A is seen as green. Nuclei were stained with DAPI (blue). The anti-Golgi marker to protein 58K was purchased from Sigma-Aldrich (St. Louis, Mo.) and was detected with a secondary antibody that can be visualized as red. Results are shown in FIG. 6. The first row of images served as a control and shows the localization of ATP7A to the Golgi. In the second row, it is observed that when B16F10 mouse melanoma cells are incubated with Cu⁺², ATP7A relocalizes away from the Golgi. In the third row, it is observed that, in the absence of stimulation with Cu⁺², SCH-28080 had no effect on ATP7A trafficking; however, when cells were incubated for 16 hours in 100 μM SCH-28080 followed by a 3 hour incubation in 10 μM Cu⁺², SCH-28080 can be seen to be a very potent inhibitor of Cu+²-stimulated ATP7A re-localization, as indicated in the images in the fourth row.

### Example IV

### Supplementation of Copper to the Omeprazole-Pretreated Cells Rescues Tyrosinase Activity/Melanin Production

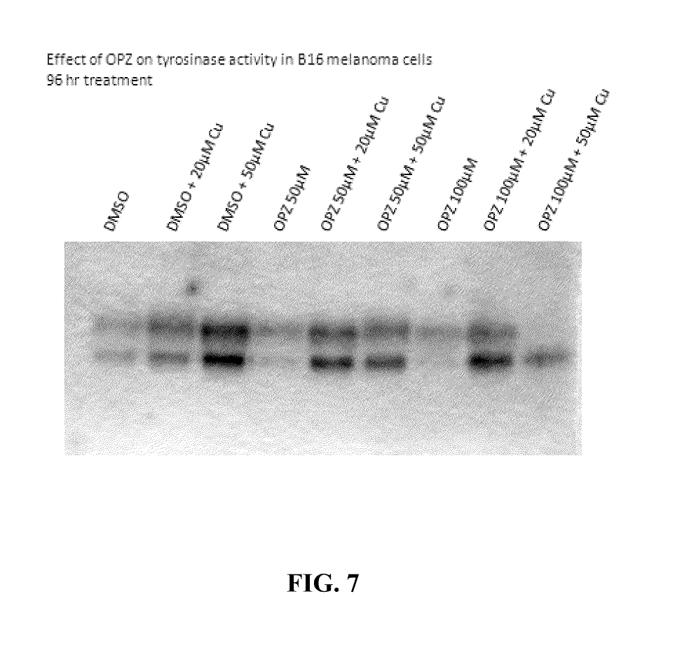

The inventors next investigated whether Cu+²-supplementation of cells could rescue tyrosinase activity that has been inhibited by incubating the cells in omeprazole. B16F10 mouse melanoma cells were incubated in omeprazole with or without additional Cu⁺² for 96 hours and then harvested. A 96 hour incubation was performed due to the possible requirement for protein turnover in order to observe omeprazole inhibition of de novo tyrosinase metallation. Protein lysates without reducing agents were fractionated using (7.5%) SDS-PAGE, and tyrosinase activity was detected colorimetrically by immersion of gels in a solution containing L-DOPA and 3-methyl-2-benzothiazolinone hydrazone. Results are shown in FIG. 7. A Western gel separation of proteins followed by in situ measurement of tyrosinase activity in B16F10 mouse melanoma cells, demonstrate that Cu⁺² can rescue cells from low levels of omeprazole. Tyrosinase activity is lower in protein extracted from cells treated with 50 μM and 100 μM omeprazole (as compared with cells treated only with Cu⁺²), but tyrosinase activity/melanin production is “rescued” by the supplementation of copper to the omeprazole-pretreated cells. Thus, exogenous copper addition to cells, at high enough concentrations such that copper enters the Golgi via low affinity pathways, appears to activate tyrosinase in the absence of ATP7A. These data strongly suggested to the inventors that the mechanism of inhibition of tyrosinase by omeprazole may be the result of a failure to incorporate copper into the tyrosinase enzyme.

### Example V

### Inhibitory Effect of Omeprazole on ATP7A Trafficking Occurs After Prolonged Pretreatment Prior to Addition of Copper

The inventors next sought to determine the length of the omeprazole incubation period needed to bring about omeprazole inhibition of ATP7A trafficking, and concluded that a treatment period of at least 72 hours was required, as 24 hour and 48 hour incubations of cells in omeprazole failed to inhibit ATP trafficking. The inventors found no evidence of trafficking inhibition, after a 1 hour pretreatment of cells with 1-100 μM omeprazole, followed by a 2 hour challenge with 50 μM Cu⁺² to elicit ATP7A trafficking to the plasma membrane. The inhibitory effect of omeprazole was observed only to occur with a prolonged (overnight) pretreatment prior to addition of copper. Further, no inhibition was observed to occur with a 30-minute pre-treatment with omeprazole prior to Cu⁺² stimulation (data not shown). This is consistent with the idea that omeprazole is metabolized to generate an inhibitory metabolite (such as hydroxy-omeprazole or perhaps the sulfonated metabolite) which then interacts with newly synthesized tyrosinase. This finding also is consistent with the hypothesis that omeprazole inhibits metallation of newly synthesized tyrosinase via ATP7A, rather than functioning as a direct inhibitor of tyrosinase per se; that is, a 72 hour incubation in omeprazole appears to be required so that preexisting tyrosinase can be turned over, allowing the inhibitory effect of omeprazole to be detected on newly synthesized tyrosinase.

### Example VI

### Omeprazole Blocks the Cu⁺²-Dependent Trafficking of ATP7B

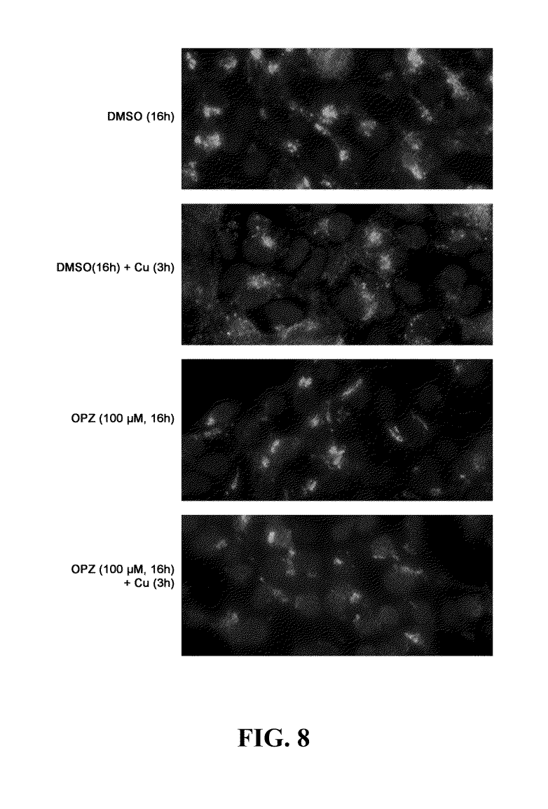

The inventors next sought to determine whether omeprazole would have any effect on the trafficking of ATP7B, and thus, potentially could be used to reverse the resistance of cancer cells to cisplatin therapy. Unlike ATP7A, ATP7B has no function in pigmentation, so the inventors assessed the possible effect of omeprazole on ATP7B trafficking patterns in cells constructed to express a tagged form of ATP7B. Human HepG2 (hepatoma) cells were stably transfected with a myc-tagged form of ATP7B and exposed overnight (16 hours) to 100 μM omeprazole dissolved in DMSO or to DMSO alone. Cu⁺² (100 μM) was then provided to cells for 3 hours to induce relocalization of ATP7B into cytoplasmic vesicles. Cells were fixed, permeabilized and probed with anti-myc antibodies to detect ATP7B. Nuclei were labeled with DAPI (blue). Results are shown in FIG. 8. Under basal conditions, ATP7B is located in the perinuclear region in the Golgi and associated vesicles (row 1). Cu+² stimulates the trafficking of ATP7B into cytoplasmic vesicles throughout the cytoplasm (row 2). Incubation of cells in 100 μM omeprazole, in the absence of Cu⁺²-stimulation, does not modify ATP7B from baseline (row 3). 100 μM omeprazole blocks this Cu+²-dependent trafficking (row 4). These results confirm that omeprazole also inhibits the trafficking of the closely related Cu+² transporter, ATP7B. Unlike ATP7A, ATP7B has no function in pigmentation, so no change in melanin synthesis is expected. This suggests that the mechanism of omeprazole inhibition involves a feature that is common to both of these Cu+²-transporting P-type ATPases, likely the conserved transmembrane 6 cysteines. In fact, omeprazole caused a marked contraction of ATP7B to a tight perinuclear location in the Golgi region consistent with inhibition of even basal levels of ATP7B trafficking.

### Example VII

### Skin-Lightening Composition

A skin-lightening silicone-in-water lotion composition, prepared in accordance with the present invention, is shown below in Table I.

TABLE I
SKIN-LIGHTENING COMPOSITION

| | |
|---|---|
| MATERIAL | WEIGHT PERCENT |

Phase I

| | |
|---|---|
| Water/phenyl trimethicone/dicapryl carbonate/ | 51.0000 |
| cimethicone/phospholipids | |
| Sodium dehydroacetate | 0.1000 |
| Disodium EDTA | 0.1400 |

Phase II

| | |
|---|---|
| Glycerin | 3.0000 |
| Omeprazole | 0.0035 |
| Aluminum starch octenylsuccinate | 1.0000 |

Phase III

| | |
|---|---|
| Purified water | 40.8065 |
| Acrylates/C10-30 alkyl acrylate crosspolymer | 0.3000 |
| Carbomer | 0.3500 |

Phase IV

| | |
|---|---|
| Glycerin | 1.0000 |
| Xanthan gum | 0.2000 |

Phase V

| | |
|---|---|
| Purified water | 2.0000 |
| Triethanolamine | 0.1000 |
| TOTAL | 100.0000 |

Procedure: In main kettle, Phase I ingredients were heated to 60° C. and mixed until uniform. In a separate kettle, Phase II ingredients were pre-mixed until uniform and then added to the main kettle. Phase III ingredients were pre-mixed until uniform and then added into the main kettle. Phase IV ingredients were pre-mixed until uniform and then added into the main kettle. The batch in main kettle was mixed with a homogenizing mixer for 15 minutes while maintaining the temperature at 60° C. Phase V ingredients were pre-mixed until clear. The batch in the main kettle was cooled to 30° C. Phase V ingredients were added to the batch in the main kettle and the batch mixed until uniform. The final pH of the batch was 5.35.

### Conclusions

The inventors' experimental data thus support their hypothesis that inhibitors of Type I H+, K+-ATPases, the 2-pyridylmethylsulfinyl-benzimidazoles and substituted imidazopyridines, are also inhibitors of P-type ATPase (ATP7A and ATP7B) intracellular trafficking and delivery of copper to copper-dependent enzymes, such as tyrosinase, an essential enzyme which is expressed in the epidermal melanocytes and which catalyzes the early steps of melanin biosynthesis. Taking advantage of published observations that localization of ATP7A to the melanosome allows for copper to be supplied to tyrosinase, the inventors demonstrated that blocking the ATP7A-mediated delivery of copper to tyrosinase, using small molecule inhibitors of ATP7A, prevents tyrosinase activation and reduces melanin synthesis. Thus, the inhibition of ATP7A trafficking by omeprazole, and its analogues, and structurally related compounds, such as SCH-28080, as shown by the inventors, may have direct consequences on melanin reduction in skin. Additionally, as it is known that overexpression of ATP7A has been associated with Alzheimer's disease, small molecule inhibitors of Cu+-ATPases may have benefits in the management and/or treatment of this disease.

Additionally, the inventors take note that cisplatin binds to ATP7B (Dimitriev, 2011), and they now claim that cisplatin binds to the same sites on the ATP7B as does copper. The inventors theorize that, since copper binds to ATP7B at the copper-binding domains (i.e., the invariant cysteines), and omeprazole also binds at the same sites, omeprazole, and its analogues, (and structurally related substituted imidazopyridines, such as SCH-28080, which bind to ATP7A/ATP7B near the cysteines) may be used to block cisplatin binding to ATP7A and/or ATP7B, and thus block sequestration or extrusion of the drug, allowing the drug to bind to tumor cell DNA, thus reversing or preventing cisplatin resistance in tumor cells.

In contrast, there are reports that cisplatin sensitivity of some tumor cells is regulated by Na+, K+-ATPase activity (Ahmed et al., 2009) rather than by copper-transporting P-type ATPases such as ATP7A and ATP7B. Studies on the effect of pH on the resistance of tumors to anti-cancer drugs (Luciani et al., 2004; DeMelito, et al., 2005) suggested, based on the observation that, while the extracellular pH of normal cells is neutral and the intracellular pH is weakly acidic, the extracellular pH of tumor cells is acidic and the intracellular pH is weakly acidic, that the regulation of cellular pH is important for tumor growth, and therefore inhibitors of the activity of vacuolar H+-ATPases (V—H+-ATPases) which are responsible for Na+/H+ exchange activity and maintaining a low pH in many cellular compartments, may reverse tumor resistance. It was suggested that PPIs, such as omeprazole, which are weak bases that accumulate in acidic compartments, and which are activated through protonation, exert antineoplastic effects on solid tumors by inhibiting V—H+-ATPase activity, thus restoring anti-cancer drug (e.g., cisplatin) sensitivity to such tumors, when used as a pretreatment. Udelnow, et al., 2011, observed the effects of omeprazole on pancreatic cancer cells and suggested that omeprazole inhibits pancreatic cancer cell proliferation and enhances the cytostatic effects of anti-cancer drugs by interacting with the regulatory functions of the V—H+-ATPase. It was hypothesized that interaction of omeprazole with the V—H+-ATPase can affect the fusion of lysosomes with autophagosomes without inhibiting its pump function. According to the teachings in the literature, PPIs should only be used in cases where a V—H+-ATPase is present.

On the other hand, the inventors now claim that pretreatment with omeprazole, before cisplatin therapy of cancer patients, whose tumors lack ATP4A, thereby loading ATP7A and ATP7B with omeprazole, would overcome cisplatin resistance by preventing cisplatin from binding to these enzymes.

To date, to the Applicants' knowledge, there has been no suggestion in the literature of the Applicants' discovery that PPIs, such as omeprazole and its analogues, and structurally related compounds, such as SCH-28080, inhibit the Cu-ATPases, ATP7A and/or ATP7B, nor that the binding of those compounds to the Cu-ATPases, ATP7A and/or ATP7B, could inhibit their trafficking between subcellular locations, nor block the binding of copper ions to these proteins, and specifically, that the binding of PPIs, such as omeprazole, to ATP7A in melanosomes, could prevent copper ion binding on the ATP7A and thus reduce pigmentation. Additionally, while the literature teaches that alteration of cellular pH is a mechanism responsible for multidrug resistance by tumor cells, the literature does not disclose or suggest that tumor cells lacking ATP4A can be treated with these small molecule inhibitors, nor do they disclose the treatment of Alzheimer's disease using a regimen including treatment with these small molecule inhibitors of ATP7A and/or ATP7B.

### REFERENCES

1. Ahmed Z, Deyama Y, Yoshimura Y, Suzuki K. Cisplatin sensitivity of oral squamous carcinoma cells is regulated by Na+, K+-ATPase activity rather than copper-transporting P-type ATPases, ATP7A and ATP7B. Cancer Chemother Pharmacol. 2009; 63:643-650.
2. Barnes N, Tsivkovskii R, Tsivkovskaia N, Lutsenko S. The copper-transporting ATPases, Menkes and Wilson disease proteins, have distinct roles in adult and developing cerebellum. J Biol Chem. 2005; 280:96940-9645.
3. Bartee, M Y, Lutsenko S. Hepatic copper-transporting ATPase ATP7B: function and inactivation at the molecular and cellular level. Biometals 2007; 20:627-637.
4. Boal A K, Rosenzweig A C. Crystal structures of cisplatin bound to a human copper chaperone. J Am chem. Soc. 2009; 131(40):14196-14197.
5. D'Amico F, Skarmoutsou E, Sanfilippo S, Camakaris J. Menkes protein localization in rat parotid acinar cells. Acta Histochem. 2005; 107: 373-378.
6. DeMilito A, Fais S. Proton pump inhibitors may reduce tumour resistance. Expert Opin. Pharmacother. 2005; 6(7): 1049-1054.
7. DiDonato M, Narindrasorasak, S Forbes J R, Cox D W, Sarkar B. Expression, purification, and metalbinding properties of the N-terminal domain from the Wilson disease putative copper-transporting ATPase (ATP7B). J Biol Chem. 1997; 272(52):33279-33282.
8. Dmitriev O, Tsivkovskii R, Abildgaard F, Morgan C T, Markley J L, Lutsenko S. solution structure of the N-domain of Wilson disease protein: distinct nucleotide-binding environment and effects of disease mutation. Proc Natl Acad Sci U.S.A. 2006; 103(14):5302-5307.
9. Dmitriev O Y. Mechanism of tumor resistance to cisplatin mediated by the copper transporter ATP7B. Biochem Cell Biol. 2011; April; 89(2):138-47.
10. Francis M J, Jones E E, Levy E R, Martin R L, Ponnambalam S, Monaco A P. Identification of a di-leucine motif within the C terminus domain of the Menkes disease protein that mediates endocytosis from the plasma membrane. J Cell Sci. 1999; 112(Pt 11):1721-1732.
11. Greenough M, Pase L, Voskoboinik I, Petris J J, O'Brien A W, Camakaris J. Signals regulating trafficking of Menkes (MNK; ATP7A) copper-translocating P-type ATPase in polarized MDCK cells. Am J Physiol Cell Physiol. 2004; 287:C1463-C1471.
12. Guo Y, Nyasae L, Braiterman L T, Hubbard A L. NH2-terminal signals in ATP7B Cu-ATPase mediate its Cu-dependent anterograde traffic in polarized hepatic cells. Am J Physiol Gastrointest Liver Physiol. 2005; 289:G904-G916.
13. Hamza I, Faisst A, Prohaska J, Chen J, Gruss P, Gitlin J D. The metallochaperone Atox 1 plays a critical role in perinatal copper homeostasis. Proc Natl Acad Sci USA. 2001; 98:6848-6852.
14. Harris E D. Cellular copper transport and metabolism. 2000; Annu Rev Nutr. 20: 291-310.
15. Hung Y H, Layton M J, Voskoboinik I, Mercer J F, Camakaris J. Purification and membrane reconstitution of catalytically active Menkes copper-transporting P-type ATPase (MNK; ATP7A). Biochem. J. 2007; 401:569-579.
16. Kodama H, Murata Y, Kobayashi M. Clinical manifestations and treatment of Menkes disease and its variants. Pediatr Int. 1999; 41:423-429.
17. Kalayda G V, Wagner C H, Buβ I, Reedijk J, Jaehde U. Altered localization of the copper efflux transporters ATP7A and ATP7B associated with cisplatin resistance in human ovarian carcinoma cells. BMC Cancer. 2008; 8(1):175.
18. Komatsu M, Sumizawa T, Mutoh M, Chen Z S, Terada K, Furukawa T. Copper-transporting P-type adenosine triphosphatase (ATP7B) is associated with cisplatin resistance. Cancer Res. 2000; 60(5):1312-1316.
19. Lane C, Petris M J, Benmerah A, Greenough M, Camakaris J. Studies on endocytic mechanism of the Menkes copper-translocating P-type ATPase (ATP7A, MNK). Endocytosis of the Menkes protein. Biometals. 2004; 17:87-98.
20. Leonhardt K, Gebhardt R, Mossner J, Lutsenko S, Huster D. Biochemical basis of regulation of human copper-transporting ATPases. Arch Biochem Biophys. 2009; 463(2): 134-148.
21. Luciani F, Spada M, De Milito A, Molinari A, Rivotini L, Montinaro A, Marra M, Lugini L, Logozzi M, Lozupone F, Federici C, Iessi E, Parmianai G, Arancia G, Belardelli F, Fais S. Effect of Proton Pump Inhibitor Pretreatment on Resistance of Solid Tumors to Cytotoxic Drugs. Journal of the National Cancer Institute. 2004; 96(22):1702-1713.
22. Lutsenko S, Petrukhin K, Cooper M J, Gilliam C T, Kaplan J H. N-terminal domains of human copper-transporting adenosine triphosphatases (the Wilson's an Menkes disease proteins) bind copper selectively in vivo and in vitro with stoichiometry of one copper per metal-binding repeat. J. Bio Chem. 1997; 272(30): 18939-18944.
23. Lutsenko S, LeShane E S, Shinde U. Biochemical basis of regulation of human copper-transporting ATPases. Arch Biochem Biophys. 2007b; 463(2): 134-148.
24. Lutsenko S, Gupta, A, Burkhead J, Zuzel V. Cellular multitasking: The dual role of human Cu-ATPases in cofactor delivery and intracellular copper balance. Arch Biochem Biophys. 2008; 476(1): 22-32.
25. Mandal A K, Arguello J M. Functional roles of metal binding domains of the Archaeoglobus flugidus Cu(+)-ATPase CopA. Biochemistry. 2003; 42:11040-11047.
26. Mercer J F, Barnes N, Stevenson J, Strausak D, Llamos R M. copper-induced trafficking of the Cu-ATPases: a key mechanism for copper homeostasis. Biometals. 2003; 16:175-184.
27. Nakagawa T, Inoue Y, Kodama H, Yamazaki H, Kawai K, Suemizu H. Expression of copper-transporting P-type adenosine triphosphatase (ATP7B) correlates with cisplatin resistance in human non-small cell lung cancer xenografts. Oncol. Rep. 2008; 20(2):265-270.
28. Petris M J, Mercer J F. The Menkes protein (ATP7A; MNK) cycles via the plasma membrane both in basal and elevated extracellular copper using a C-terminal di-leucine endocytic signal. Hum Mol Genet. 1999; 8:2107-2115.
29. Petris M J, Strausak D, Mercer J F. The Menkes copper transport is required for the activation of tyrosinase. 2000; Hum Mol Genet. 9: 2845-2851.
30. Ralle M, Lutsenko S, Blackburn J J. Copper transfer to the N-terminal domain of the Wilson disease protein (ATP7B): X-ray absorption spectroscopy of reconstituted and chaperone-loaded metal bind domains and their interaction with exogenous ligands. J Inorg Bioch. 2004: 98:765-774.
31. Samimi G, Safaei R, Katano, K, Holzer A K, Rochdi M, Tomioka M. Increased expression of the copper efflux transporter ATP7A mediates resistance to cisplatin, carboplatin and oxaliplatin in ovarian cancer cells. Clin Cancer Res. 2004; 10(14):4661-4669.
32. Samimi G, Safaei R, Katano, K, Holzer A K, Safaei R, Howell S B. Modulation of the cellular pharmacology of cisplatin and its analogs by the copper exporters ATP7A and ATP7B. Mol Pharmacol. 2004; 53:13-23.
33. Schallreuter K U, Rokos H. From the bench to the bedside: proton pump inhibitors can worsen vitiligo. British Journal of Dermatology 2007; 156:1371-1373.
34. Shaefer M, Gitlin J D. Genetic disorders of membrane transport. IV. Wilson's disease and Menkes disease. Am J Physiol. 1999; 276:G311-G314.
35. Thiele D J. Integrating trace element metabolism from the cell to the whole organism. J Nutr. 2003; 133: 15795-15805.
36. Udelnow A, Kreyes A, Ellinger S, Landfester K, Walther P, Klapperstueck T, Wohlrab J, Henne-Bruns D, Knkipschild U and Würl P. Omeprazole Inhibits Proliferation and Modulates Autophagy in Pancreatic Cancer Cells. PloS ONE. 2011; 6(5):1-17.
37. Voskoboinik I, Strausak D, Greenough M, Brooks H, Petris M, Smith S, Mercer J F, Camakaris J. functional analysis of the N-terminal CXXC metal-binding motifs in the human Menkes copper-transporting P-type ATPase expressed in cultured mammalian cells. J. Biol Chem. 1992; 274:22008-22012.
38. Voskoboinik I, Greenough M, La Fontaine S, Mercer J F, Camakaris J. Functional studies on the Wilson copper P-type ATPase and toxic milk mouse mutant. Biochem Biophys Res Commun. 2001; 281: 966-970.
39. Wang N, Daniels R, Hebert D N. The cotranslational maturation of the type I membrane glycoprotein tyrosinase: the heat shock protein 70 system hands off to the lectin-based chaperone system. Mol Biol Cell. 2005; 16(8):3740-52.
40. Wernimont A K, Huffman D L, Lamb A L, O'Halloran T V, Rosenzweig A C. Structural basis for copper transfer by the metallochaperone for the Menkes/Wilson disease proteins. Nat Struct Biol. 2000; 7(9):766-771.
41. Wilson S K. Progressive lenticular degeneration: a familial nervous disease associated with cirrhosis of the liver. Brain. 1912; 34:295-507.
42. Yamaguchi Y, Heiny, M E, Suzuki M, Gitlin J D. Biochemical characterization and intracellular localization of the Menkes disease protein. 1996; Proc. Natl Acad Sci USA; 93: 14030-14035.
43. Yatsunyk L A, Rosenzweig A C. Cu(I) binding and transfer by the N terminus of the Wilson disease protein. J Biol Chem. 2007; 282(12):8622-8631.
44. Yoshizawa K, Nozaki S, Kitahara H, Ohara T, Kato K, Kawashiri S, Yamamoto E. Copper efflux transporter (ATP7B) contributes to the acquisition of cisplatin-resistance in human oral squamous cell lines. Oncol. Rep. 2007; 18(4):987-991.
45. Zeynep T, Moller L. Menkes disease. Eur. J. Hum. Genet. 2010; May 18(5):511-518.
46. Zheng Z, White C, Lee J, Peterson, T S, Bush A I, Sun G. Y, Weisman G A and Petris M J. Altered Microglial copper homeostasis in a mouse model of Alzheimer's disease. Journal of Neurochemistry. 2010; 114:1630-1638.

While the subject invention has been described in various preferred embodiments, the skilled artisan will appreciate that various modifications, substitutions, omissions and changes may be made without departing from the spirit and scope of the invention recited in the following claims.

We claim:

1. A small molecule inhibitor of ATP7A or ATP7B having the structural formula: 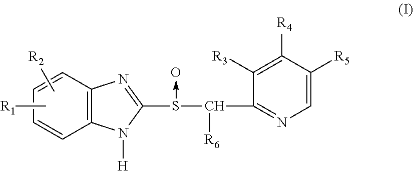
wherein:
R₁ and R₂ are same or different and are each selected from the group consisting of hydrogen, alkyl, carbomethoxy, carboethoxy, alkoxy, and alkanoyl, any of which may be halogen-substituted, and halogen;
R₆ is selected from the group consisting of hydrogen, methyl, and ethyl; and
R₃, R₄ and R₅ are the same or different and are each selected from the group consisting of hydrogen, methyl, methoxy, ethoxy, methoxyethoxy, ethoxyethoxy, propoxy, propoxymethoxy, and the like, any of which may be halogen-substituted; or a derivative or physiologically acceptable salt, solvate or bioprecursor, or stereoisomer or enantiomer thereof;
or having the structural formula: 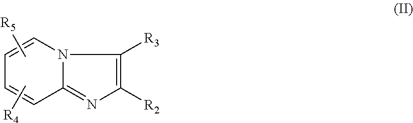
wherein:
R₂ is hydrogen, lower alkyl or hydroxy lower alkyl;
R₃ is lower alkyl, —CH₂CN, hydroxy lower alkyl, —NO, —CH₂N═C or 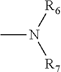
(wherein R₆ and R₇ are independently selected from the group consisting of hydrogen and lower alkyl) or hydrogen provided R₂ is not hydrogen;
R₄ is Z-T-W wherein Z represents —O—, —NH— or a single bond; T represents a straight- or branched-chain lower-alkylene group; when Z is a single bond, T also represents an ethenylene or a propenylene group wherein the unsaturated carbon is at the single bond; when Z is —O—, T also represents an allylene group wherein the saturated carbon is at the oxygen; and W represents hydrogen, when T is allylene and Z is —O—, and Ar, wherein Ar is selected from thienyl, pyridinyl, furanyl, phenyl and substituted phenyl wherein there are one or more substituents on the phenyl independently selected from halogen or lower alkyl; and
R₅ is hydrogen, halogen or lower alkyl; and a topically applicable, cosmetically or dermatologically acceptable vehicle, carrier or diluent therefor; or a derivative or physiologically acceptable salt, solvate or bioprecursor, or stereoisomer or enantiomer thereof.

2. The small molecule inhibitor of ATP7A or ATP7B, according to claim 1, which is selected from the group consisting of omeprazole, 5- or 6-methoxy-2-{[(4-methoxy-3,5-dimethylpyridin-2-yl)methyl]sulfinyl}-1H-benzimidazole; esomeprazole, S-5-methoxy-2-{(4-methoxy-3,5 dimethylpyridin-2-yl)methylsufinyl]-3H-benzoimidazole; lansoprazole, 2-{[3-methyl-4-(2,2,2-trifluoroethoxy)pyridin-2-yl]methylsulfinyl-1H-benzo(d)imidazole; pantoprazole, RS-6-(difluoromethoxy))-2-[(3,4-dimethoxypyridin-2-yl)methylsulfinyl]-1H-benzo(d)imidazole; rabeprazole (pariprazole), 2-([4-(3-methoxypropoxy)-3-methylpyridin-2-yl]methylsulfinyl)-1H-benzo(d)imidazole, leminoprazole, 2-((o-(isobutylmethylamino)benzyl)sulfinyl)benzimidazole; and timoprazole, 2-(pyridine-2-ylmethylsulfinyl)-1H-benzimidazole.

3. The small molecule inhibitor of ATP7A or ATP7B according to claim 2, which comprises omeprazole, 5- or 6-methoxy-2-{[(4-methoxy-3,5-dimethylpyridin-2-yl)methyl]sulfinyl}-1H-benzimidazole.

4. The small molecule inhibitor of ATP7A or ATP7B according to claim 1, having the formula:

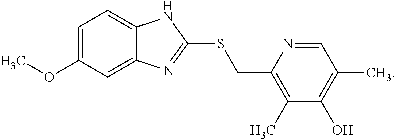 [SMILES: COc1ccc2[nH]c(SCc3ncc(C)c(O)c3C)nc2c1]

5. The small molecule inhibitor of ATP7A or ATP7B according to claim 1, having the formula:

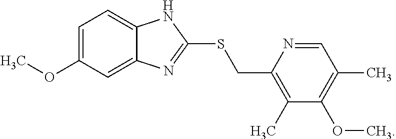 [SMILES: COc1ccc2[nH]c(SCc3ncc(C)c(OC)c3C)nc2c1]

6. The small molecule inhibitor of ATP7A or ATP7B according to claim 1, which is selected from the group consisting of SCH-28080; soraprazan [(7R,8R,9R)-2,3-dimethyl-8-hydroxy-7(2-methoxyethoxy)-9-phenyl-7,8,9,10-tetrahydro-imidazo-[1,2-h][1,7]-naphthyridine], pumaprazole 8-(2-methoxycarbonylamino-6-benzulamino)-2,3-dimethylimidazo-[1,2-a)pyridine-D,L-hemimalate, AR-H047108, (8-[(2-ethyl-6-methylbenzyl)amino]2,3-dimethylimidazo[1,2-a]pyridine-6-carboxamide; dapiprazole, 3-{2-[4-(2-methylphenyl)piperazin-1-yl]ethyl}-5,6,7,8-tetrahydro-[1,2,4]triazolo[4,5-a]pyridine; AZD0865, ((8-[2,6-dimethylbenzyl)amino]-N-(2-hydroxyethyl)-2,3-dimethylimidazo[1,2-a]pyridine-6-carboxamide; and tenatoprazole, 3-methoxy-8-[(4-methoxy-3,5-dimethyl-pyridin-2-yl)methylsulfinyl]-2,7,9-triazabicyclo[4.3.0]nona-2,4,8,10-tetraene.

7. The small molecule inhibitor of ATP7A or ATP7B according to claim 6, which comprises SCH-28080.

8. A cosmetic, dermatological and/or pharmaceutical composition comprising a small molecule inhibitor of ATP7A and/or ATP7B, according to claim 1, in a cosmetically-, dermatologically- or pharmaceutically-acceptable vehicle, carrier or diluent therefor.

9. A cosmetic, dermatological and/or pharmaceutical composition comprising a small molecule inhibitor of ATP7A and/or ATP7B, according to claim 2, in a cosmetically-, dermatologically- or pharmaceutically-acceptable vehicle, carrier or diluent therefor.

10. A cosmetic, dermatological and/or pharmaceutical composition comprising a small molecule inhibitor of ATP7A and/or ATP7B, according to claim 3, in a cosmetically-, dermatologically- or pharmaceutically-acceptable vehicle, carrier or diluent therefor.

11. A cosmetic, dermatological and/or pharmaceutical composition comprising a small molecule inhibitor of ATP7A and/or ATP7B, according to claim 4, in a cosmetically-, dermatologically- or pharmaceutically-acceptable vehicle, carrier or diluent therefor.

12. A cosmetic, dermatological and/or pharmaceutical composition comprising a small molecule inhibitor of ATP7A and/or ATP7B, according to claim 5, in a cosmetically-, dermatologically- or pharmaceutically-acceptable vehicle, carrier or diluent therefor.

13. A cosmetic, dermatological and/or pharmaceutical composition comprising a small molecule inhibitor of ATP7A and/or ATP7B, according to claim 6, in a cosmetically-, dermatologically- or pharmaceutically-acceptable vehicle, carrier or diluent therefor.

14. A cosmetic, dermatological and/or pharmaceutical composition comprising a small molecule inhibitor of ATP7A and/or ATP7B, according to claim 7, in a cosmetically-, dermatologically- or pharmaceutically-acceptable vehicle, carrier or diluent therefor.

15. A method of inhibiting ATP7A and/or ATP7B in a subject, other than by affecting a V—H+-ATPase, the method comprising administering to the subject in need thereof a composition comprising a small molecule inhibitor of ATP7A and/or ATP7B having the structural formula: 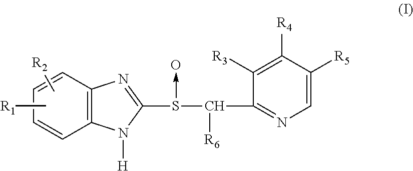
wherein:
R₁ and R₂ are same or different and are each selected from the group consisting of hydrogen, alkyl, carbomethoxy, carboethoxy, alkoxy, and alkanoyl, any of which may be halogen-substituted, and halogen;
R₆ is selected from the group consisting of hydrogen, methyl, and ethyl; and
R₃, R₄ and R₅ are the same or different and are each selected from the group consisting of hydrogen, methyl, methoxy, ethoxy, methoxyethoxy, ethoxyethoxy, propoxy, propoxymethoxy, and the like, any of which may be halogen-substituted; or a derivative or physiologically acceptable salt, solvate or bioprecursor, or stereoisomer or enantiomer thereof;
or having the structural formula: 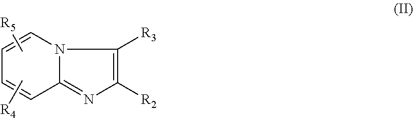
wherein:
R₂ is hydrogen, lower alkyl or hydroxy lower alkyl;
R₃ is lower alkyl, —CH₂CN, hydroxy lower alkyl, —NO, —CH₂N═C, hydrogen (provided R₂ is not hydrogen), or 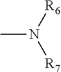
(wherein R₆ and R₇ are independently selected from the group consisting of hydrogen and lower alkyl);
R₄ is Z-T-W wherein Z represents —O—, —NH— or a single bond; T represents a straight- or branched-chain lower-alkylene group; when Z is a single bond, T also represents an ethenylene or a propenylene group wherein the unsaturated carbon is at the single bond; when Z is —O—, T also represents an allylene group wherein the saturated carbon is at the oxygen; and W represents hydrogen, when T is allylene and Z is —O—, and Ar, wherein Ar is selected from thienyl, pyridinyl, furanyl, phenyl and substituted phenyl wherein there are one or more substituents on the phenyl independently selected from halogen or lower alkyl; and
R₅ is hydrogen, halogen or lower alkyl; or a derivative or physiologically acceptable salt, solvate or bioprecursor, or stereoisomer or enantiomer thereof; and
a topically applicable, cosmetically, dermatologically- or pharmaceutically-acceptable vehicle, carrier or diluent therefor.

16. The method according to claim 15, wherein the small molecule inhibitor of ATP7A or ATP7B is selected from the group consisting of omeprazole, 5- or 6-methoxy-2-{[(4-methoxy-3,5-dimethylpyridin-2-yl)methyl]sulfinyl}-1H-benzimidazole; esomeprazole, S-5-methoxy-2-{(4-methoxy-3,5 dimethylpyridin-2-yl)methylsufinyl]-3H-benzoimidazole; lansoprazole, 2-{[3-methyl-4-(2,2,2-trifluoroethoxy)pyridin-2-yl]methylsulfinyl-1H-benzo(d)imidazole; pantoprazole, RS-6-(difluoromethoxy))-2-[(3,4-dimethoxypyridin-2-yl)methylsulfinyl]-1H-benzo(d)imidazole; rabeprazole (pariprazole), 2-([4-(3-methoxypropoxy)-3-methylpyridin-2-yl]methylsulfinyl)-1H-benzo(d)imidazole, leminoprazole, 2-((o-(isobutylmethylamino)benzyl)sulfinyl)benzimidazole; and timoprazole, 2-(pyridine-2-ylmethylsulfinyl)-1H-benzimidazole.

17. The method according to claim 16, wherein the small molecule inhibitor of ATP7A or ATP7B comprises omeprazole, 5- or 6-methoxy-2-{[(4-methoxy-3,5-dimethylpyridin-2-yl)methyl]sulfinyl}-1H-benzimidazole.

18. The method according to claim 15, wherein the small molecule inhibitor of ATP7A or ATP7B has the formula:

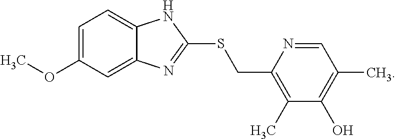 [SMILES: COc1ccc2[nH]c(SCc3ncc(C)c(O)c3C)nc2c1]

19. The method according to claim 15, wherein the small molecule inhibitor of ATP7A or ATP7B has the formula:

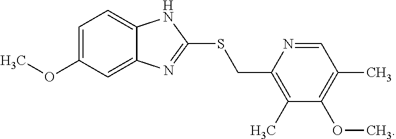 [SMILES: COc1ccc2[nH]c(SCc3ncc(C)c(OC)c3C)nc2c1]

20. The method according to claim 15, wherein the small molecule inhibitor of ATP7A or ATP7B comprises SCH-28080; soraprazan [(7R,8R,9R)-2,3-dimethyl-8-hydroxy-7(2-methoxyethoxy)-9-phenyl-7,8,9,10-tetrahydro-imidazo-[1,2-h][1,7]-naphthyridine], pumaprazole 8-(2-methoxycarbonylamino-6-benzulamino)-2,3-dimethylimidazo-[1,2-a)pyridine-D,L-hemimalate, AR-H047108, (8-[(2-ethyl-6-methylbenzyl)amino]2,3-dimethylimidazo[1,2-a]pyridine-6-carboxamide; dapiprazole, 3-{2-[4-(2-methylphenyl)piperazin-1-yl]ethyl}-5,6,7,8-tetrahydro-[1,2,4]triazolo[4,5-a]pyridine; AZD0865, ((8-[2,6-dimethylbenzyl)amino]-N-(2-hydroxyethyl)-2,3-dimethylimidazo[1,2-a]pyridine-6-carboxamide; and tenatoprazole, 3-methoxy-8-[(4-methoxy-3,5-dimethyl-pyridin-2-yl)methylsulfinyl]-2,7,9-triazabicyclo[4.3.0]nona-2,4,8,10-tetraene.

21. The method according to claim 20, wherein the small molecule inhibitor of ATP7A or ATP7B comprises SCH-28080.

22. A method of inhibiting melanogenesis in skin cells of a subject, other than by affecting a V—H+-ATPase, the method comprising administering to the subject in need thereof a composition comprising a small molecule inhibitor of ATP7A according to claim 1, in a cosmetically-, dermatologically- or pharmaceutically-acceptable vehicle, carrier or diluent therefor.

23. A method of inhibiting the level of copper ions in cells, other than by a method of chelating or binding the copper ions, the method comprising treating the cells with a composition comprising a small molecule inhibitor of ATP7A or ATP7B, according to claim 1, in a cosmetically-, dermatologically- or pharmaceutically-acceptable vehicle, carrier or diluent therefor.

24. A method of prophylaxis or treatment of Alzheimer's disease, by reversing up-regulation of ATP7A and thereby reducing excretion of copper ions by ATP7A in cells of a subject, other than by affecting a V—H+-ATPase, the method comprising the step of administering to the subject in need thereof a composition comprising a prophylactically- or therapeutically-effective amount of small molecule inhibitor of ATP7A or ATP7B, according to claim 1, in a pharmaceutically-acceptable vehicle, carrier or diluent therefor.

25. A method of overcoming anti-cancer drug-resistance in tumor cells of a subject, other than by affecting a V—H+-ATPase, the method comprising the step of administering to the subject in need thereof, a composition comprising a small molecule inhibitor of ATP7A and/or ATP7B, according to claim 1, in a cosmetically-, dermatologically- or pharmaceutically-acceptable vehicle, carrier or diluent therefor.

26. The method according to claim 25, wherein the anti-cancer drug is cisplatin.

27. The method according to claim 25, which comprises overcoming the anti-cancer drug-resistance in tumor cells without increasing pigmentation in the skin cells of the subject.

28. A molecule formed by reacting a 2-pyridylmethylsulfinyl-benzimidazole with a small, low molecular weight compound containing a sulfhydryl functional group under acidic pH conditions.

29. The molecule according to claim 28, wherein the 2-pyridylmethylsulfinyl-benzimidazole is selected from the group consisting of omeprazole, 5- or 6-methoxy-2-{[(4-methoxy-3,5-dimethylpyridin-2-yl)methyl]sulfinyl}-1H-benzimidazole; esomeprazole, S-5-methoxy-2-{(4-methoxy-3,5 dimethylpyridin-2-yl)methylsufinyl]-3H-benzoimidazole; lansoprazole, 2-{[3-methyl-4-(2,2,2-trifluoroethoxy)pyridin-2-yl]methylsulfinyl-1H-benzo(d)imidazole; pantoprazole, RS-6-(difluoromethoxy))-2-[(3,4-dimethoxypyridin-2-yl)methylsulfinyl]-1H-benzo(d)imidazole; rabeprazole (pariprazole), 2-([4-(3-methoxypropoxy)-3-methylpyridin-2-yl]methylsulfinyl)-1H-benzo(d)imidazole, leminoprazole, 2-((o-(isobutylmethylamino)benzyl)sulfinyl)benzimidazole; and timoprazole, 2-(pyridine-2-ylmethylsulfinyl)-1H-benzimidazole.

30. The molecule according to claim 28, wherein the small, low molecular weight compound containing a sulfhydryl functional group is selected from the group consisting of L-cysteine, L-cysteamine, 2-mercaptoethanol and glutathione.

31. The molecule according to claim 28, having the formula:

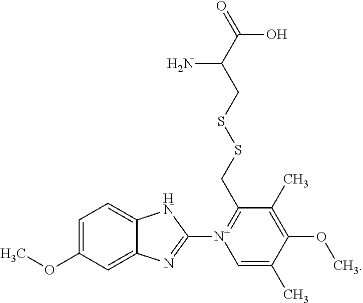 [SMILES: COc1ccc2[nH]c(-[n+]3cc(C)c(OC)c(C)c3CSSCC(N)C(=O)O)nc2c1]

32. The molecule according to claim 28, having the formula:

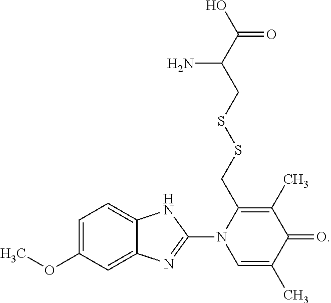 [SMILES: COc1ccc2[nH]c(-n3cc(C)c(=O)c(C)c3CSSCC(N)C(=O)O)nc2c1]

33. The molecule according to claim 28, having the formula:

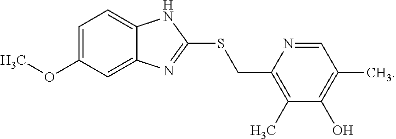 [SMILES: COc1ccc2[nH]c(SCc3ncc(C)c(O)c3C)nc2c1]

34. The molecule according to claim 28, having the formula:

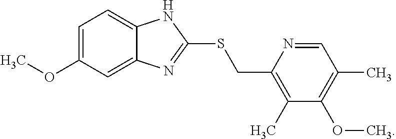 [SMILES: COc1ccc2[nH]c(SCc3ncc(C)c(OC)c3C)nc2c1]

35. A method of inhibiting melanogenesis in skin cells of a subject, other than by affecting a V—H+-ATPase, the method comprising administering to the subject in need thereof a composition comprising the molecule of claim 28, in a cosmetically-, dermatologically- or pharmaceutically-acceptable vehicle, carrier or diluent therefor.

36. A method of inhibiting melanogenesis in skin cells of a subject, other than by affecting a V—H+-ATPase, the method comprising administering to the subject in need thereof a composition comprising the molecule of claim 33, in a cosmetically-, dermatologically- or pharmaceutically-acceptable vehicle, carrier or diluent therefor.

37. A method of inhibiting melanogenesis in skin cells of a subject, other than by affecting a V—H+-ATPase, the method comprising administering to the subject in need thereof a composition comprising the molecule of claim 34, in a cosmetically-, dermatologically- or pharmaceutically-acceptable vehicle, carrier or diluent therefor.


SEQUENCE LISTING (3 sequences)

SEQ ID NO: 1 - protein, 60 aa, Homo sapiens
KSIAYTLTKNIPELTPYLIYITVSVPLPLGCITILFIELCTDIFPSVSLAYEKAESDIMH

SEQ ID NO: 2 - protein, 108 aa, Homo sapiens
LGFEASLVKKDRSASHLDHKREIRQWRRSFLVSLFFCIPVMGLMIYMMVMDHHFATLHHNQNMSKEEMINLHSSMFLERQILPQLSVMNLLSFLLCVPLEFFGGWYFY

SEQ ID NO: 3 - protein, 86 aa, Homo sapiens
FVSIATLLVWIVIGFLNFEIVETYFPGYNRSISRTETIIRFAFQASITVLCIACPCSLGLATPTAVMVGTGVGAQNGILIKGGEPL